SEQUENCE LISTING (37 sequences)

SEQ ID NO: 1 - DNA, 1733 bp, Artificial or Unknown
  CAG
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattg

SEQ ID NO: 2 - DNA, 134 bp, Artificial or Unknown
  NFAT promoter
ggaggaaaaactgtttcatacagaaggcgtggaggaaaaactgtttcatacagaaggcgtggaggaaaaactgtttcatacagaaggcgtagatctagactctagagggtatataatggaagctcgaattccag

SEQ ID NO: 3 - DNA, 66 bp, Artificial or Unknown
  P2A
ggaagcggagccacgaacttctctctgttaaagcaagcaggagatgttgaagaaaaccccgggcct

SEQ ID NO: 4 - DNA, 587 bp, Artificial or Unknown
  IRES
cccctctccctcccccccccctaacgttactggccgaagccgcttggaataaggccggtgtgcgtttgtctatatgttattttccaccatattgccgtcttttggcaatgtgagggtccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaacc

SEQ ID NO: 5 - DNA, 48 bp, Artificial or Unknown
  CL1
gcctgcaagaactggttcagcagcctgagccacttcgtgatccacctt

SEQ ID NO: 6 - DNA, 522 bp, Artificial or Unknown
  ployA
tcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 7 - DNA, 720 bp, Artificial or Unknown
  EGFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcactcagtccgccctgagcaaagaccccaacgagaagcgcgatcacatggtcctgctggagttcgtgaccgccgccgggatcactctcggcatggacgagctgtacaagtaa

SEQ ID NO: 8 - DNA, 237 bp, Artificial or Unknown
  GLP1(7-37)
atgaagatcatcctgtggctgtgtgtgttcggcctgttcctggccaccctgttccccatcagctggcagatgcccgtggagtccggcctgtcctccgaggactccgccagctccgagagcttcgccaagcgcatcaagcgccacgccgagggcacctttaccagcgacgtgtcctcttacctggaaggccaggccgccaaagagtttatcgcctggctggtcaagggcagaggctga

SEQ ID NO: 9 - DNA, 234 bp, Artificial or Unknown
  GLP1(7-37)mutant1
atgaagatcatcctgtggctgtgtgtgttcggcctgttcctggccaccctgttccccatcagctggcagatgcccgtggagtccggcctgtcctccgaggactccgccagctccgagagcttcgccaagcgcatcaagcgccacggcgagggcacctttaccagcgacgtgtcctcttacctggaaggccaggccgccaaagagtttatcgcctggctggtcaagggcagaggc

SEQ ID NO: 10 - DNA, 234 bp, Artificial or Unknown
  GLP1(7-37)mutant2
atgaagatcatcctgtggctgtgtgtgttcggcctgttcctggccaccctgttccccatcagctggcagatgcccgtggagtccggcctgtcctccgaggactccgccagctccgagagcttcgccaagcgcatcaagcgccacggagaagggacgttcactagtgacgtcagctcttaccttgaaggtcaagccgcgcaggaattcattgcgtggctcgttgatggcaggggg

SEQ ID NO: 11 - DNA, 258 bp, Artificial or Unknown
  Extendin 4
atgaagatcatcctgtggctgtgtgtgttcggcctgttcctggccaccctgttccccatcagctggcagatgcccgtggagtccggcctgtcctccgaggactccgccagctccgagagcttcgccaagcgcatcaagcgccacggcgaaggaactttcacctctgacctctccaaacaaatggaggaagaagctgtacgactcttcatcgaatggctcaaaaacggtggaccatcaagcggggctccgcccccttcc

SEQ ID NO: 12 - DNA, 198 bp, Artificial or Unknown
  HCRT 1-66
atgaaccttccttccacaaaggtctcctgggccgccgtgacgctactgctgctgctgctgctgctgccgcccgcgctgttgtcgtccggggcggctgcacagcccctgcccgactgctgtcgtcaaaagacttgctcttgccgcctctacgagctgctgcacggcgcgggcaatcacgcggccggcatcctcacgctg

SEQ ID NO: 13 - DNA, 210 bp, Artificial or Unknown
  HCRT 1-33 Furin HCRT 34-66
atgaacctgcccagtaccaaggtgagctgggccgcggtcacccttttgctgcttctgctcctgctcccccccgctctgctcagttcaggagctgctgcccgcatcaagcgccagcccctgcccgactgctgtcgtcaaaagacttgctcttgccgcctctacgagctgctgcacggcgcgggcaatcacgcggccggcatcctcacgctg

SEQ ID NO: 14 - DNA, 240 bp, Artificial or Unknown
  Ext4-SP Furin HCRT 34-66
atgaagatcatcctgtggctgtgtgtgttcggcctgttcctggccaccctgttccccatcagctggcagatgcccgtggagtccggcctgtcctccgaggactccgccagctccgagagcttcgccaagcgcatcaagcgccagcccctgcccgactgctgtcgtcaaaagacttgctcttgccgcctctacgagctgctgcacggcgcgggcaatcacgcggccggcatcctcacgctg

SEQ ID NO: 15 - DNA, 168 bp, Artificial or Unknown
  mSA Furin HCRT 34-66
atgaaatgggtaacattcattagtcttctttttctcttctcaagcagctctcgcgcccgcatcaagcgccagcccctgcccgactgctgtcgtcaaaagacttgctcttgccgcctctacgagctgctgcacggcgcgggcaatcacgcggccggcatcctcacgctg

SEQ ID NO: 16 - DNA, 393 bp, Artificial or Unknown
  HCRT 1-131
atgaaccttccttccacaaaggtctcctgggccgccgtgacgctactgctgctgctgctgctgctgccgcccgcgctgttgtcgtccggggcggctgcacagcccctgcccgactgctgtcgtcaaaagacttgctcttgccgcctctacgagctgctgcacggcgcgggcaatcacgcggccggcatcctcacgctgggcaagcggaggtccgggcccccgggcctccagggtcggctgcagcgcctcctgcaggccagcggcaaccacgccgcgggcatcctgaccatgggccgccgcgcaggcgcagagccagcgccgcgcccctgcctcgggcgccgctgttccgccccggccgccgcctccgtcgcgcccggaggacagtccgggatc

SEQ ID NO: 17 - DNA, 213 bp, Artificial or Unknown
  ACTH-S
atgaagatcatcctgtggctgtgtgtgttcggcctgttcctggccaccctgttccccatcagctggcagatgcccgtggagtccggcctgtcctccgaggactccgccagctccgagagcttcgccaagcgcatcaagcgctcctactccatggagcacttccgctggggcaagccggtgggcaagaagcggcgcccagtgaaggtgtaccct

SEQ ID NO: 18 - DNA, 264 bp, Artificial or Unknown
  ACTH-L
gccaccatgaagatcatcctgtggctgtgtgtgttcggcctgttcctggccaccctgttccccatcagctggcagatgcccgtggagtccggcctgtcctccgaggactccgccagctccgagagcttcgccaagcgcatcaagcgctcctactccatggagcacttccgctggggcaagccggtgggcaagaagcggcgcccagtgaaggtgtaccctaacggcgccgaggacgagtcggccgaggccttccccctggagttc

SEQ ID NO: 19 - DNA, 579 bp, Artificial or Unknown
  Leptin-Ex
atgaagatcatcctgtggctgtgtgtgttcggcctgttcctggccaccctgttccccatcagctggcagatgcccgtggagtccggcctgtcctccgaggactccgccagctccgagagcttcgccaagcgcatcaagcgcgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgc

SEQ ID NO: 20 - DNA, 501 bp, Artificial or Unknown
  Leptin-FL
atgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgc

SEQ ID NO: 21 - DNA, 1434 bp, Artificial or Unknown
  hMelanopsin
atgaaccctcctagcggccctagagttcctccatctcctacacaagagcccagctgcatggccacaccagctccaccatcttggtgggatagcagccagagcagcatcagcagtctggggagactgcctagcatcagccctacagcacctggaacatgggctgctgcttgggtgccactgcctacagtggatgtgcctgatcacgcccactacaccctgggcacagtgattctgctcgtgggcctgactggcatgctgggcaatctgaccgtgatctacaccttctgccggtccagaagcctgagaacccctgccaacatgttcatcatcaacctggccgtgtccgacttcctgatgagcttcacacaggcccctgtgttcttcaccagcagcctgtacaagcagtggctgttcggcgagacaggctgcgagttctacgccttttgtggcgccctgttcggcatcagctccatgatcaccctgaccgctatcgccctggacagatacctggtcatcaccagacctctggccacctttggcgtggcctctaagagaagggccgcctttgttctgctcggcgtgtggctgtatgccctggcttggagtctgcctccattctttggctggagcgcctatgtgccagagggcctgctgacaagctgtagctgggactacatgtccttcacaccagccgtgcgggcctacaccatgctgctgtgctgtttcgtgttcttcctgcctctgctgatcatcatctactgctacatcttcatcttccgggccatcagagagacaggcagagccctgcagacattcggcgcctgtaaaggcaatggcgagagcctgtggcagcggcagagactccagtctgagtgcaagatggccaagatcatgctgctcgtgatcctgctgttcgtgctgagctgggccccttattctgctgtggctctggtggcctttgccggctatgctcatgtgctgaccccttacatgagcagcgtgccagccgtgattgccaaggccagcgccatccacaatcctatcatctacgccatcacacaccccaagtacagagtggcaatcgcccagcatctgccttgtctgggagtgctgctgggagtgtccagaaggcacagcagaccctatcctagctaccgcagcacccacagaagcaccctgacaagccacacctccaacctgagctggatcagcatccggcggagacaagagtctctgggcagcgagtctgaagtcggctggacccatatggaagccgctgctgtttggggagctgcacagcaggccaatggcagatctctgtatggccagggcctcgaggatctggaagccaaggctcctcctagacctcagggacacgaagccgagacacctggcaagaccaagggactgatccccagccaggatcctagaatg

SEQ ID NO: 22 - DNA, 7104 bp, Artificial or Unknown
  dCas9VPR
atggatgctttagacgattttgacttagatatgcttggttcagacgcgttagacgacttcgacctagacatgttaggctcagatgcattggacgacttcgatttagatatgttgggctccgatgccctagatgactttgatctagatatgctaggtagtcccaaaaagaagaggaaagtgcccgggcagtatctgcccgacacagatgatagacaccgaatcgaagagaaacgcaagcgaacgtatgaaaccttcaaatcgatcatgaagaaatcgcccttctcgggtccgaccgatcccaggcccccaccgagaaggattgcggtcccgtcccgctcgtcggccagcgtgccgaagcctgcgccgcagccctaccccttcacgtcgagcctgagcacaatcaattatgacgagttcccgacgatggtgttcccctcgggacaaatctcacaagcctcggcgctcgcaccagcgcctccccaagtccttccgcaagcgcctgccccagcgcctgcaccggcaatggtgtccgccctcgcacaggcccctgcgcccgtccccgtgctcgcgcctggaccgccccaggcggtcgctccaccggctccgaagccgacgcaggccggagagggaacactctccgaagcacttcttcaactccagtttgatgacgaggatcttggagcactccttggaaactcgacagaccctgcggtgtttaccgacctcgcgtcagtagataactccgaatttcagcagcttttgaaccagggtatcccggtcgcgccacatacaacggagcccatgttgatggaataccccgaagcaatcacgagacttgtgacgggagcgcagcggcctcccgatcccgcacccgcacctttgggggcacctggcctccctaacggacttttgagcggcgacgaggatttctcctccatcgccgatatggatttctcagccttgctgtcacagatttccagcggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggacccagcgcccgcagtgactcccgaggccagtcacctgttggaggaccccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttcatatgtcaagcatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccactagtgtgatcaagcccgacatgaaaatcaagctgaggatggagggtgccgtgaacggtcacccgtttgcgatcgagggcgtgggactgggcaagccgttcgagggaaagcaaagcatcgacctcaaggtgaaggagggcggaccgctgcccttcgcctacgacattctgaccactgccttctgctacggtaatcgcgtcttcgcaaagtatcccgaaaacatcgtggattactttaagcagagcttcccggagggttactcctgggagcgatccatgaactacgaggacggagggatctgcaacgcgacgaacgatataaccttggacggcgactgctacatttacgaaattcggttccgcggcaccaatttcccagcgaatggccctgtaatgcaaaaacgcacggtaaagtgggagcccagcaccgagaacctgtacgtccgcgacggagtcctgaagggagacgtgatcatggcgctttcattggaaggtggcgggcactacagatgcgatttcaagacgacctataaggcgaaaaaggtggtccaactgccggactaccatttcgtcgaccaccacatcgagatcaagagccacgacaaagactatagcaacgtgaatctccacgagcatgccgaggcccacagcgagctgcccaggcaggccaagactagtgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggatccgtgattaaaccagacatgaagatcaagctgcgtatggaaggcgctgtaaatggacacccgttcgcgattgaaggagttggccttgggaagcctttcgagggaaaacagagtattgaccttaaagtcaaagaaggcggacctctgcctttcgcctatgacatcttgacaactgctttctgttacggcaacagggtattcgccaaatacccagaaaatatagtagactatttcaagcagtcgtttcctgagggctactcttgggaacgaagcatgaattacgaagacgggggcatttgtaacgcgacaaacgacataaccctggatggtgactgttatatctatgaaattcgatttagaggtacgaactttcctgccaatggtccagttatgcagaagaggactgtgaaatgggagccatccactgagaacttgtatgtgcgtgatggagtgctgaagggtgatgttatcatggctctgtcgcttgaaggaggtggccattaccgatgtgacttcaaaactacttataaagctaagaaggttgtccagttgccagactatcactttgtggaccaccacattgagattaaaagccacgacaaagattacagtaatgttaatctgcatgagcacgccgaagcgcattctgagctgccgaggcaggccaagggatcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaagctagcggctcccccaagaaaaaacgcaaggtgtaa

SEQ ID NO: 23 - DNA, 588 bp, Artificial or Unknown
  CMV
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 24 - DNA, 600 bp, Artificial or Unknown
  Puro
atgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 25 - DNA, 795 bp, Artificial or Unknown
  Neo
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 26 - DNA, 942 bp, Artificial or Unknown
  SOUL
atggactatggcggcgctttgtctgccgtcggacgcgaacttttgttcgttactaatcctgtggtggtgaacgggtccgtcctggtccctgaggatcaatgttactgtgccggatggattgaatctcgcggcacgaacggcgctcagaccgcgtcaaatgtcctgcagtggcttgcagcaggattcagcattttgctgctgatgttctatgcctaccaaacctggaaatctacatgcggctgggaggagatctatgtgtgcgccattgaaatggttaaggtgattctcgagttcttttttgagtttaagaatccctctatgctctaccttgccacaggacaccgggtgcagtggctgcgctatgcagagtggctgctcacttctcctgtcatccttatccacctgagcaacctcaccggcctgagcaacgactacagcaggagaaccatgggactccttgtctcagccatcgggtgtatcgtgtggggggctaccagcgccatggcaaccggctatgttaaagtcatcttcttttgtcttggattgtgctatggcgcgaacacattttttcacgccgccaaagcatatatcgagggttatcatactgtgccaaagggtcggtgccgccaggtcgtgaccggcatggcatggctgtttttcgtgagctggggtatgttcccaattctcttcattttggggcccgaaggttttggcgtcctgagcgtctatggctccaccgtaggtcacacgattattgatctgatgagtaaaaattgttgggggttgttgggacactacctgcgcgtcctgatccacgagcacatattgattcacggagatatccgcaaaaccaccaaactgaacatcggcggaacggagatcgaggtcgagactctcgtcgaagacgaagccgaggccggagccgtgccagcggccgcctga

SEQ ID NO: 27 - DNA, 942 bp, Artificial or Unknown; identical to SEQ ID NO: 26
  SOUL(L132C)

SEQ ID NO: 28 - DNA, 1560 bp, Artificial or Unknown
  SEAP
atgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatga

SEQ ID NO: 29 - DNA, 5300 bp, Artificial or Unknown
  Plasmid Construction
cctcgacctgcagcccaagcttcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcgccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctcgagttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttg

SEQ ID NO: 30 - DNA, 479 bp, Artificial or Unknown
  PIR3_HSP70min promoter
gaaatagcgctgtacagcgtatgggaatctcttgtacggtgtacgagtatcttcccgtacacgtagaaatagcgctgtacagcgtatgggaatctcttgtacggtgtagagtatcttcccgtacaccgtacgaaatagcgctgtacagcgtatggaatctcttgtacggtgtacgagtatcttcccgtacaccgtacgccggagtataaatagaggcgcttcgtctacggagcgacaattcaattcaaacaagcaaagtgaacacgtcgctaagcgaaagctaagcaaataaacaagcgcagctgaacaagctaaacaatctgcagtaaagtgcaagttaaagtgaatcaattaaaagtaaccagcaaccaagtaaatcaactgcaactactgaaatctgccaagaagtaattattgaatacaagaagagaactctgaatactttcaacaagttaccgagaaagaagaactcacacaca

SEQ ID NO: 31 - DNA, 702 bp, Artificial or Uknown
  PIR3HSP70min Promoter
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtga

SEQ ID NO: 32 - DNA, 462 bp, Artificial or Unknown
  IL4
atgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctga

SEQ ID NO: 33 - DNA, 2091 bp, Artificial or Uknown
  PhyB-VP16-NLS
atggtttccggagtcgggggtagtggcggtggccgtggcggtggccgtggcggagaagaagaaccgtcgtcaagtcacactcctaataaccgaagaggaggagaacaagctcaatcgtcgggaacgaaatctctcagaccaagaagcaacactgaatcaatgagcaaagcaattcaacagtacaccgtcgacgcaagactccacgccgttttcgaacaatccggcgaatcagggaaatcattcgactactcacaatcactcaaaacgacgacgtacggttcctctgtacctgagcaacagatcacagcttatctctctcgaatccagcgaggtggttacattcagcctttcggatgtatgatcgccgtcgatgaatccagtttccggatcatcggttacagtgaaaacgccagagaaatgttagggattatgcctcaatctgttcctactcttgagaaacctgagattctagctatgggaactgatgtgagatctttgttcacttcttcgagctcgattctactcgagcgtgctttcgttgctcgagagattaccttgttaaatccggtttggatccattccaagaatactggtaaaccgttttacgccattcttcataggattgatgttggtgttgttattgatttagagccagctagaactgaagatcctgcgctttctattgctggtgctgttcaatcgcagaaactcgcggttcgtgcgatttctcagttacaggctcttcctggtggagatattaagcttttgtgtgacactgtcgtggaaagtgtgagggacttgactggttatgatcgtgttatggtttataagtttcatgaagatgagcatggagaagttgtagctgagagtaaacgagatgatttagagccttatattggactgcattatcctgctactgatattcctcaagcgtcaaggttcttgtttaagcagaaccgtgtccgaatgatagtagattgcaatgccacacctgttcttgtggtccaggacgataggctaactcagtctatgtgcttggttggttctactcttagggctcctcatggttgtcactctcagtatatggctaacatgggatctattgcgtctttagcaatggcggttataatcaatggaaatgaagatgatgggagcaatgtagctagtggaagaagctcgatgaggctttggggtttggttgtttgccatcacacttcttctcgctgcataccgtttccgctaaggtatgcttgtgagtttttgatgcaggctttcggtttacagttaaacatggaattgcagttagctttgcaaatgtcagagaaacgcgttttgagaacgcagacactgttatgtgatatgcttctgcgtgactcgcctgctggaattgttacacagagtcccagtatcatggacttagtgaaatgtgacggtgcagcatttctttaccacgggaagtattacccgttgggtgttgctcctagtgaagttcagataaaagatgttgtggagtggttgcttgcgaatcatgcggattcaaccggattaagcactgatagtttaggcgatgcggggtatcccggtgcagctgcgttaggggatgctgtgtgcggtatggcagttgcatatatcacaaaaagagactttcttttttggtttcgatctcacactgcgaaagaaatcaaatggggaggcgctaagcatcatccggaggataaagatgatgggcaacgaatgcatcctcgttcgtcctttcaggcttttcttgaagttgttaagagccggagtcagccatgggaaactgcggaaatggatgcgattcactcgctccagcttattctgagagactcttttaaagaatctgaggcggctatgaactctaaagttgtggatggtgtggttcagccatgtagggatatggcgggggaacaggggattgatgagttaggtgacgcccttgacgattttgacttagacatgctcccagccgatgcccttgacgactttgaccttgatatgctgcctgctgacgctcttgacgattttgaccttgacatgctccccgccgccaagagagtgaagctggactga

SEQ ID NO: 34 - DNA, 810 bp, Artificial or Unknown
  PIF6
atgatgagtcgaggagaggtgcgcatggcgaaggcagggcgggaggggccgcgggacagcgtgtggctgtcgggggaggggcggcgcggcggtcgccgtgggcggcagccgtccgggctcgaccgggaccggatcaccggggtcaccgtccggctgctggacacggagggcctgacggggttctcgatgcaccgcctggccgccgagctgaacgtcaccgcgatgtccgtgtactggtacgtcgacaccaaggaccagttgctcgagctcgccctggacgccgtcttcggcgagctgcgccacccggacccggacgccgggctcgactggcgcgaggaactgcgggccctggcccgggagaaccgggcgctgctggtgcgccacccctggtcgtcccggctggtcggcacctacctcaacatcggcccgcactcgctggccttctcccgcgcggtgcagaacgtcgtgcgccgcagcgggctgcccgcgcaccgcctgaccggcgccatctcggccgtcttccagttcgtctacggctacggcaccatcgagggccgcttcctcgcccgggtggcggacaccgggctgagtccggaggagtacttccaggactcgatgaccgcggtgaccgaggtgccggacaccgcgggcgtcatcgaggacgcgcaggacatcatggcggcccggggcggcgacaccgtggcggagatgctggaccgggacttcgagttcgccctcgacctgctcgtcgcgggcatcgacgcgatggtcgaacaggcctacccatacgatgttccagattacgcttga

SEQ ID NO: 35 - DNA, 915 bp, Artificial or Unknown
  EL222
atgccaaaaaagaagagaaaggtagcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggggaattcggcgccgacgacacccgggtggaagtgcagcctcctgcccagtgggtgctggacctgatcgaggccagccctatcgccagcgtggtgtccgaccctagactggccgacaaccccctgatcgccatcaaccaggccttcaccgatctgaccggctacagcgaagaggaatgcgtgggccggaactgccggtttctggccggctctggcaccgagccctggctgaccgacaagatcagacagggcgtgcgcgagcacaagcccgtgctggtggaaatcctgaactacaagaaggacggcacccccttccggaacgctgtgctggtggcccccatctacgacgacgacgatgagctgctgtacttcctgggcagccaggtggaagtggacgacgaccagcccaacatgggcatggccagacgcgagcgggctgccgagatgctgaaaaccctgagccccagacagctggaagtgaccaccctggtggccagcggcctgcggaacaaagaggtggccgccagactgggcctgagcgagaaaaccgtgaagatgcaccggggcctggtcatggaaaagctgaacctgaaaaccagcgccgacctcgtgcggatcgccgtggaagccggcatcccaaaaaagaagagaaaggtatga

SEQ ID NO: 36 - DNA, 190 bp, Artificial or Uknown
  C120 promoter
taggtagcctttagtccatgcgttataggtagcctttagtccatgcgttataggtagcctttagtccatgcgttataggtagcctttagtccatgcgttataggtagcctttagtccatgaagcttagacactagagggtatataatggaagctcgacttccagcttggcaatccggtactgttggtaaa

SEQ ID NO: 37 - DNA, 363 bp, Artificial or Unknown
  BDNF
atgcactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatag

(19) United States
(12) Patent Application Publication
(10) Pub. No.: US 2024/0254507 A1
(43) Pub. Date: Aug. 1, 2024

(54) ENGINEERED CELLS FOR PRODUCING OF THERAPEUTIC AGENTS TO BE DELIVERED BY A HYBRID BIOELECTRONIC DEVICE

(71) Applicants: NORTHWESTERN UNIVERSITY, Evanston, IL (US); WILLIAM MARSH RICE UNIVERSITY, Houston, TX (US)

(72) Inventors: Omid Veiseh, Bellaire, TX (US); Jacob Robinson, Houston, TX (US); Jonathan Rivnay, Chicago, IL (US); Isaac Hilton, Houston, TX (US); Samantha T. Fleury, Houston, TX (US); Matthew C. Parker, Houston, TX (US); Jacob H. Goell, Houston, TX (US); Jing Li, Houston, TX (US); Kaiyuan Wang, Houston, TX (US)

(21) Appl. No.: 18/287,709

(22) PCT Filed: Apr. 21, 2022
(86) PCT No.: PCT/US2022/025724

§ 371 Date: Oct. 20, 2023

Related U.S. Application Data
(60) Provisional application No. 63/177,806, filed on Apr. 21, 2021.

(51) Int. Cl. C12N 15/85; C07K 14/435; C12N 9/22; C12N 9/50

(52) U.S. Cl. CPC C12N 15/85; C07K 14/435; C12N 9/22; C12N 9/50; C12Y 304/22062; C12N 2830/001

(57) ABSTRACT

An engineered cell expressing a therapeutic agent and a reporter agent, the engineered cell comprising a first transgene containing a light sensing DNA sequence encoding a light sensing protein, and a second transgene containing a calcium activated promoter, a therapeutic agent DNA encoding a therapeutic agent, and a reporter agent DNA encoding a reporter agent.

### CROSS-REFERENCE TO RELATED PATENT APPLICATIONS

This application claims priority to and the benefit of U.S. Provisional Patent Application Ser. No. 63/177,806, filed Apr. 21, 2021, which is incorporated herein in its entirety by reference.

This application is also related to co-pending PCT patent applications, entitled “Hybrid Bioelectronic/Engineered Cell Implantable System For Therapeutic Agents Delivery And Applications Thereof”, with Attorney Docket No. 0116936.266WO2, and “Hybrid Bioelectronic/Engineered Cell Wearable System For Therapeutic Agents Delivery And Applications Thereof”, with Attorney Docket No. 0116936.266WO22, respectively, which are filed on the same day that this application is filed, and with the same applicant as that of this application, which are incorporated herein by reference in their entireties.

### STATEMENT AS TO RIGHTS UNDER FEDERALLY-SPONSORED RESEARCH

This invention was made with government support under FA8650-21-2-7119 awarded by the Air Force Research Laboratory. The government has certain rights in the invention.

### FIELD OF THE INVENTION

The present disclosure relates generally to field of biotechnology, and more particularly to genetically engineered cells producing therapeutic agents which are precisely delivered to a subject's body, e.g., blood stream, using a hybrid bioelectronics device/system.

### BACKGROUND OF THE INVENTION

The background description provided herein is for the purpose of generally presenting the context of the present invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions.

A key challenge for biological production of therapeutic agents is controlling the production levels, which can vary due to cell health, cell metabolism, temperature and etc. Production of desired therapeutic agents by genetically engineered cells, due to the nature of the art, is hard to be precisely controlled, and therefore, its use in medical treatments is significantly undermined.

Therefore, there remains an imperative need for genetically engineered cells producing desired therapeutic agents whose production and delivery can be precisely controlled.

### SUMMARY OF THE INVENTION

In light of the foregoing, this invention discloses genetically engineered cells, when using with a hybrid bioelectronic device, producing desired therapeutic agents whose production and delivery can be precisely controlled. The engineered cells in combination with the hybrid bioelectronic device contains a low-power bioelectronic feedback control system based on an optogenetic system in the engineered cells and fluorescent tracking of therapy production levels. Opsins in the optogenetic system enables a low-power control signal. A second innovation is the bioelectronic feedback loop based on fluorescent tracking of the production levels. The engineered cells are created to produce a fluorescent protein at a fixed ratio (e.g., near 1:1) relative to the therapy. Using this fluorescence measurement as the feedback signal, one can regulate the on time of the cell factories to maintain a stable fixed point of therapy production with precision that exceeds synthetic biological feedback loops.

This work establishes a generalizable engineered cells/implantable device framework to precisely control and deliver therapeutic agents to a subject's body.

In one aspect of the invention, an engineered cell expressing a therapeutic agent and a reporter agent, the engineered cell comprising a first transgene comprising a light sensing protein DNA sequence encoding a light sensing protein; a second transgene comprising a light sensing protein activated promoter, a therapeutic agent DNA encoding a therapeutic agent, and a reporter agent DNA encoding a reporter agent; a third transgene comprising a light sensing protein activated promoter and a dCas9 DNA encoding a dCas9 protein; and a fourth transgene comprising a CASP9 DNA encoding an iCaspase9 protein leading to cell death; wherein the light sensing protein, when receiving a light signal, activates the light sensing protein activated promoter; wherein the light sensing protein activated promoter is configured to drive production of the therapeutic agent and the reporter agent; and wherein the therapeutic agent and the reporter agent are configured to be expressed at a substantially fixed ratio.

In one embodiment, the light sensing protein comprises at least one of a step-function opsin (SOUL), a mutant of SOUL, a melanopsin, a PhyB/PIF6, a PhyB/PIF3, and a EL222.

In one embodiment, the light sensing protein DNA sequence comprises one of SEQ ID Nos: 21, 26-27, and 33-35 or an equivalent DNA of one of SEQ ID Nos: 21, 26-27, and 33-35.

In one embodiment, the light sensing protein activated promoter comprises one of SEQ ID Nos: 2, 30, and 36 or an equivalent DNA of one of SEQ ID No: 2, 30, and 36.

In one embodiment, the therapeutic agent DNA comprises one of SEQ ID Nos: 8-20, 28, 31, 32 and 37 or an equivalent DNA of SEQ ID Nos: 8-20, 28, 31, 32, and 37.

In one embodiment, the reporter agent DNA comprises SEQ ID No: 7 or an equivalent DNA of SEQ ID No: 7.

In one embodiment, the second transgene comprises an IRES DNA or a P2A DNA locates between the therapeutic agent DNA and the reporter agent DNA.

In one embodiment, the IRES DNA comprises SEQ ID No: 4 or an equivalent DNA of SEQ ID No: 4, and the P2A DNA comprises SEQ ID No: 3 or an equivalent DNA of SEQ ID No: 3.

In one embodiment, the engineered cell is an ARPE-19 cell transfected by the first, second, third, and fourth transgenes.

In one embodiment, the engineered cell is a HEK293T cell transfected by the first, second, third, and fourth transgenes.

In one embodiment, the engineered cell is derived from a cell line comprising MCF-12a cells, or hTERT-MSCs.

In one embodiment, the engineered cell is derived from primary cells comprising islets or MSCs.

In another aspect of the invention, an engineered cell expressing a therapeutic agent and a reporter agent, the engineered cell comprising a first transgene comprising an light sensing protein DNA sequence encoding an light sensing protein; and a second transgene comprising a light sensing protein activated promoter, a therapeutic agent DNA encoding a therapeutic agent, and a reporter agent DNA encoding a reporter agent.

In one embodiment, the light sensing protein comprises one of a step-function opsin (SOUL), a mutant of SOUL, a melanopsin, a PhyB/PIF6, a PhyB/PIF3, and a EL222.

In one embodiment, the light sensing protein activated promoter in the engineered cell, upon activation by the light sensing protein, drives production of the therapeutic agent and the reporter agent.

In one embodiment, the light sensing protein DNA sequence comprises one of SEQ ID Nos: 21, 26-27, and 33-35 or an equivalent DNA of one of SEQ ID Nos: 21, 26-27, and 33-35.

In one embodiment, the light sensing protein activated promoter is one of a NFAT promoter comprising SEQ ID No: 2 or an equivalent DNA of SEQ ID No: 2, a PIR3_HSP70 min promoter comprising SEQ ID No: 30 or an equivalent DNA of SEQ ID No: 30, and a C120 promoter comprising SEQ ID No: 36 or an equivalent DNA of SEQ ID No: 36.

In one embodiment, the therapeutic agent and the reporter agent are expressed at a substantially fixed ratio.

In one embodiment, the therapeutic agent DNA comprises one of SEQ ID Nos: 8-20, 28, 31, 32, and 37 or an equivalent DNA of one of SEQ ID Nos: 8-20, 28, 31, 32, and 37.

In one embodiment, the reporter agent DNA comprises SEQ ID No: 7 or an equivalent DNA of SEQ ID No: 7.

In one embodiment, the second transgene comprises an IRES DNA or a P2A DNA locates between the therapeutic agent DNA and the reporter agent DNA.

In one embodiment, the IRES DNA comprises SEQ ID No: 4 or an equivalent DNA of SEQ ID No: 4, and the P2A DNA comprises SEQ ID No: 3 or an equivalent DNA of SEQ ID No: 3.

In one embodiment, the engineered cell further comprising a third transgene comprising a light sensing protein activated promoter and a dCas9 DNA encoding a dCas9 protein.

In one embodiment, the engineered cell further comprising a fourth transgene comprising a CASP9 DNA encoding an iCaspase9 protein leading to cell death.

In one embodiment, the engineered cell is an ARPE-19 cell transfected by the first and second transgenes.

In one embodiment, the engineered cell is a HEK293T cell transfected by the first and second transgenes.

In another aspect of the invention, a method for producing an engineered cell expressing a therapeutic agent and a reporter agent, the method comprising transfecting a cell with a first transgene comprising a light sensing protein DNA sequence encoding a light sensing protein; and transfecting the cell with a second transgene containing a light sensing protein activated promoter, a therapeutic agent DNA encoding a therapeutic agent, and a reporter agent DNA encoding a reporter agent.

In one embodiment, the method further comprising transfecting the cell with a third transgene comprising a light sensing protein activated promoter and a dCas9 DNA encoding a dCas9 protein.

In one embodiment, the method further comprising transfecting the cell with a fourth transgene containing a CASP9 DNA encoding an iCaspase9 protein leading to cell death.

In one embodiment, the light sensing protein comprises one of a step-function opsin (SOUL), a mutant of SOUL, a melanopsin, a PhyB/PIF6, a PhyB/PIF3, and a EL222.

In one embodiment, the light sensing protein activated promoter is one of a NFAT promoter comprising SEQ ID No: 2 or an equivalent DNA of SEQ ID No: 2, a PIR3_HSP70 min promoter comprising SEQ ID No: 30 or an equivalent DNA of SEQ ID No: 30, and a C120 promoter comprising SEQ ID No: 36 or an equivalent DNA of SEQ ID No: 36.

In one embodiment, the therapeutic agent and the reporter agent are expressed at a substantially fixed ratio.

In one embodiment, the therapeutic agent DNA comprises one of SEQ ID Nos: 8-20, 28, 31, 32, and 37 or an equivalent DNA of SEQ ID Nos: 8-20, 28, 31, 32, and 37.

In one embodiment, the reporter agent DNA comprises SEQ ID No: 7 or an equivalent DNA of SEQ ID No: 7.

In another aspect of the invention, a transgene configured to produce a therapeutic agent and a reporter agent at a substantially fixed ratio, the transgene comprising following formula:

(Light sensing protein activated promoter)-(B)ₙ-(Therapeutic agent DNA)-(B)ₘ-(IRES DNA)-(B)ₒ-(Reporter agent DNA); or

(Light sensing protein activated promoter)-(B)ₙ-(Reporter agent DNA)-(B)ₘ-(P2A DNA)-(B)ₒ-(Therapeutic agent DNA)

wherein B is independent deoxyribonucleotide; and n, m, o are integer numbers, wherein n, m, and o can be same or different from each other.

In one embodiment, the light sensing protein activated promoter is one of a NFAT promoter comprising SEQ ID No: 2 or an equivalent DNA of SEQ ID No: 2, a PIR3_HSP70 min promoter comprising SEQ ID No: 30 or an equivalent DNA of SEQ ID No: 30, and a C120 promoter comprising SEQ ID No: 36 or an equivalent DNA of SEQ ID No: 36.

In one embodiment, the IRES DNA comprises SEQ ID No: 4 or an equivalent DNA of SEQ ID No: 4.

In one embodiment, the P2A DNA comprises SEQ ID No: 3 or an equivalent DNA of SEQ ID No: 3.

In one embodiment, therapeutic agent DNA comprises one of SEQ ID Nos: 8-20, 28, 31, 32, and 37 or an equivalent DNA of SEQ ID Nos: 8-20, 28, 31, 32, and 37.

In one embodiment, the reporter agent DNA comprises SEQ ID No: 7 or an equivalent DNA of SEQ ID No: 7.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

Illustrative embodiments of the invention will hereafter be described with reference to the accompanying drawings, wherein like numerals denote like elements.

FIGS. 1A-C provide different embodiments of hybrid bioelectronic implantable device. FIG. 1A illustrates an implantable embodiment having a single cell housing containing engineered cells. FIG. 1B illustrates an implantable embodiment having plurality of cell housings containing same or different engineered cells. FIG. 1C illustrates an implantable embodiment having one or more cell housings integrated with power transduction management system, optoelectronics and other accessary systems e.g., O₂ generation system.

FIG. 2 is a graphical depiction of the proposed synthetic biology circuit for optogenetic control of the peptide therapeutic Orexin A in accordance with an illustrative embodiment.

FIG. 3 depicts preliminary data showing that ARPE-19 cells can be made to express luciferase with high on/off ratio in response to blue light using an EL222 optogenetic system in accordance with an illustrative embodiment.

FIG. 4 shows a biohybrid precision control scheme based on co-production of therapeutic peptide and proxy reporter fluorophore (GFP*) in accordance with an illustrative embodiment.

FIGS. 5A-B illustrate validation of cellular GLP-1 production. FIG. 5A shows a schematic of GLP-1 expression plasmids. CAG: CMV enhancer, chicken beta-Actin promoter; CL1: degron; P2A: self-cleaving peptide; IRES: internal ribosome entry site; Exendin-4 SP: exendin-4 signal peptide; Furin: furin cleavage site; GLP-1(7-37)-mutant1: GLP-1(7-37)Gly8; GLP-1(7-37)-mutant2: GLP-1(7-37)Gly8/Gln26/Asp34; PA, polyA signal. FIG. 5B shows HEK 293T cells were transfected with GLP-1 vectors and the media changed 24 hrs post-transfection.

FIGS. 6A-B illustrate validation of cellular orexin-A production. FIG. 6A shows a schematic of orexin-A expression plasmids. Orexin-A constitutes amino acids 34-66 of hypocretin (HCRT) while HCRT1-33 is the original secretion signal of orexin-A. Exendin 4 SP: secretion signal peptide of exendin-4. mSA SP: modified human albumin signal peptide. Furin: furin cleavage site. HCRT 1-131: full length gene of hypocretin. FIG. 6B shows orexin-A production by HEK 293T cells 24 hours after transfection of expression plasmids.

FIGS. 7A-C illustrate validation of cellular ACTH and leptin production. FIG. 7A shows a schematic of expression plasmids of ACTH, leptin and prokinectin-2. ACTH 1-39: full ACTH gene. ACTH 1-24: a biologically active short version of ACTH. Exendin-4 sp: secretion signal peptide of exendin-4. Leptin 22-146: leptin neuropeptide. Leptin 1-21: leptin secretion signal. FIG. 7B shows ACTH production by HEK 293T cells 24 hours after transfection of expression plasmids. FIG. 7C shows leptin production by HEK293T cells 24-hours after transfection of expression plasmids.

FIGS. 8A-B show production of Leptin from clonal ARPE-19 cell lines. FIG. 8A shows a schematic of leptin expression plasmid; FIG. 8B shows leptin production by transfected ARPE-19 cells.

FIGS. 9A-B show validation of 1:1 correlation of Leptin and GFP production in ARPE-19 cells lines. FIG. 9A shows correlation of GFP and leptin expression on the protein level. FIG. 9B shows mRNA levels of leptin and EGFP which were quantified using qPCR.

FIGS. 10A-B show production of ACTH from clonal ARPE-19 cell lines, FIG. 10A shows a schematic of ACTH expression plasmid ACTH: Adrenocorticotropic hormone. FIG. 10B shows ACTH production of the transfected ARPE-19 cells.

FIGS. 11A-B show validation of 1:1 correlation of ACTH and GFP production in ARPE-19 cells lines. FIG. 11A shows correlation of GFP and ACTH expression on the protein level. FIG. 11B shows mRNA levels of ACTH and EGFP which were quantified using qPCR.

FIGS. 12A-B show production of GLP-1 from clonal ARPE-19 cell lines. FIG. 12A shows a schematic of GLP-1 expression plasmid GLP-1(7-37); Glucagon Like Peptide 1. FIG. 12B shows GLP-1 production from the transfected ARPE-19 cells.

FIGS. 13A-B show validation of 1:1 correlation of GLP-1 and GFP production in ARPE-19 cells lines. FIG. 13A shows correlation of GFP and GLP-1 expression on the protein level. FIG. 13B shows mRNA levels of GLP-1 and EGFP which were quantified using qPCR.

FIGS. 14A-B show light induced production of leptin from ARPE-19 cells transfected with the melanopsin optogenetic system. FIG. 14A shows a schematic of optogenetic control plasmid and calcium responsive leptin expression plasmid NFAT: Nuclear factor of activated T-cells promoter; Neo; Neomycin resistance. FIG. 14B shows leptin production in transfected ARPE-19 cells.

FIGS. 15A-B show testing of alternative optogenetic systems. FIG. 15A shows GLP-1 mRNA level in HEK293T cells which were transfected with NFAT-GLP-1, photoswitchable dCas9-VPR, and a sgRNA targeting the NFAT promoter. FIG. 15B shows SEAP expression from HEK293 Ts transiently expressing hMelanopsin and NFAT-SEAP from cells either grown in the dark or exposed to blue light over the course of 24 hours.

FIGS. 16A-C show testing of the SOUL optogenetic system. FIG. 16A shows SEAP expression from HEK293 Ts transiently expressing SOUL and NFAT-SEAP from cells either grown in the dark or exposed to blue light over the course of 24 hours. FIG. 16B shows calcium staining in HEK-293T cells expressing SOUL, SOUL(L132C) exposed to blue light and imaged over 40 seconds. FIG. 16C shows SEAP expression from HEK293 Ts transiently expressing SOUL, SOUL(L132C) and NFAT-SEAP from cells either grown in the dark or exposed to blue light over the course of 24 hours.

FIGS. 17A-B show light induced production of leptin from ARPE-19 cells transfected with the SOUL(L132C) optogenetic system. FIG. 17A shows a schematic of optogenetic control plasmid and calcium responsive leptin expression plasmid SOUL(L132C); step-function opsin with ultra-high light sensitivity with an additional L132C mutation. FIG. 17B shows leptin expression in ARPE-19 cells which were stably transfected with the plasmids in FIG. 17A.

FIGS. 18A-B show validation of PhyB-PIF6 optogenetic system. FIG. 18A shows a schematic of expression plasmids. CAG: CMV enhancer, chicken beta-Actin promoter; PhyB: Phytochrome B; IRES: internal ribosome entry site; PIF6: Phytochrome Interacting Factor 6; PA, poly(A) signal TNF: tumor necrosis factor; IL4: Interleukin 4 Puro: puromycin resistance; Neo: neomycin resistance. FIG. 18B shows stably engineered ARPE-19 cells were plated and exposed to light or kept in the dark, and their expressions of TNF and IL4.

FIGS. 19A-B show validation of EL222 optogenetic system. FIG. 19A shows a schematic of expression plasmids. CAG: CMV enhancer, chicken beta-Actin promoter; EL222: light-oxygen-voltage domain and LuxR-type helix-turn-helix DNA-binding domain; PA, poly(A) signal; BDNF: Brain derived neurotrophic factor; Puro: puromycin resistance; Neo: neomycin resistance. FIG. 19B shows stably engineered ARPE-19 cells were plated and exposed to light or kept in the dark, and their expression of BDNF.

FIG. 20 depicts phases of peripheral and central clocks in response to an 8 hr shift, for normal entrainment (left), providing therapy affecting only the central clock (middle), and the proposed hybrid bioelectronics/engineered cells approach (right) with therapy targeting both central and peripheral clocks in accordance with an illustrative embodiment.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the invention has been specifically disclosed by preferred embodiments and optional features, modification and variation of the concepts herein disclosed may be resorted to by those skilled in the art, and that such modifications and variations are considered to be within the scope of this invention as defined by the appended claims.

Whenever a range is given in the specification, for example, a temperature range, a time range, or a composition or concentration range, all intermediate ranges and subranges, as well as all individual values included in the ranges given are intended to be included in the invention. It will be understood that any subranges or individual values in a range or subrange that are included in the description herein can be excluded from the claims herein.

It will be understood that, as used in the description herein and throughout the claims that follow, the meaning of “a”, “an”, and “the” includes plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising”, or “includes” and/or “including”, or “has” and/or “having”, or “carry” and/or “carrying”, or “contain” and/or “containing”, or “involve” and/or “involving”, “characterized by”, and the like are to be open-ended, i.e., to mean including but not limited to. When used in this disclosure, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the invention, and will not be interpreted in an idealized or overly formal sense unless expressly so defined herein.

As used in the disclosure, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in the disclosure, the phrase “at least one of A, B, and C” should be construed to mean a logical (A or B or C), using a non-exclusive logical OR. As used herein, the term “and/or” includes any and all combinations of one or more of the associated listed items.

As used in the disclosure, the term “implantable” refers to an ability of a device to be positioned at a location within a body, such as subcutaneously, within a body cavity, or etc. Furthermore, the terms “implantation” and “implanted” refer to the positioning of a device at a location within a body, such as subcutaneously, within a body cavity, or etc.

As used in the disclosure, “biocompatible” material is a material that is compatible with living tissue or a living system by not being toxic or injurious and not causing immunological rejection.

As used in the disclosure, “therapeutic agent” refers to any substance that provides therapeutic effects to a medical or mental condition, as well as physiological condition or symptom related thereto. In certain embodiments, a therapeutic agent refers to a substance that provides therapeutic effects to any diseases or biological or physiological responses. The therapeutic agent may be a biological substance, such as a nucleic acid (e.g., a nucleotide, DNA, or RNA), a polypeptide, a lipid, a sugar (e.g., a monosaccharide, disaccharide, oligosaccharide, or polysaccharide), a small molecule, etc. In some embodiments, the therapeutic agent is a polypeptide.

As used in the disclosure, the term “therapy” refers to any protocol, method, and/or agent that can be used in the management, treatment, and/or amelioration of a given disease, or a symptom related thereto. In certain embodiments, the terms “therapies” and “therapy” refer to a biological therapy, supportive therapy, and/or other therapies known to one of skill in the art, such as medical personnel, useful in the management or treatment of a given disease, or symptom related thereto.

As used in the disclosure, “treat,” “treatment,” and “treating” refer to the reduction or amelioration of the progression, severity, and/or duration of a given disease resulting from the administration of one or more therapies (including, but not limited to, the administration of microspheres disclosed herein). In certain embodiments, the terms refer to the reduction of pain associated with one or more diseases or conditions.

As used in the disclosure, “engineered cell(s)” refers herein to cells having been engineered, e.g., by the introduction of an exogenous nucleic acid sequence or specific alteration of an endogenous gene sequence. An exogenous nucleic acid sequence that is introduced may comprise a wild type sequence of any species that may be modified. An engineered cell may comprise genetic modifications such as one or more mutations, insertions and/or deletions in an endogenous gene and/or insertion of an exogenous nucleic acid (e.g., a genetic construct) in the genome. An engineered cell may refer to a cell in isolation or in culture. Engineered cells may be “transduced cells” wherein the cells have been infected with e.g., an engineered virus. For example, a retroviral vector may be used, such as described in the examples, but other suitable viral vectors may also be contemplated such as lentiviruses. Non-viral methods may also be used, such as transfections or electroporation of DNA vectors. DNA vectors that may be used are transposon vectors. Engineered cells may thus also be “stably transfected cells” or “transiently transfected cells”. Transfection refers to non-viral methods to transfer DNA (or RNA) to cells such that a gene is expressed. Transfection methods are widely known in the art, such as calcium phosphate transfection, PEG transfection, and liposomal or lipoplex transfection of nucleic acids. Such a transfection may be transient, but may also be a stable transfection wherein cells can be selected that have the gene construct integrated in their genome.

As used in the disclosure, “transgene” refers to a nucleic acid sequence (encoding, for example, a therapeutic agent or a reporter agent) that is partly or entirely heterologous, i.e., foreign, to the host cell into which it is introduced. The transgene is inserted into an organism, host cell, or vector in a manner that ensures its function.

As used in the disclosure, “vector” or “plasmid” refers to discrete elements that are used to introduce heterologous nucleic acids or transgenes into cells for either expression of the heterologous nucleic acid or for replication of the heterologous nucleic acid. One vector may contain one or more transgenes. Selection and use of such vectors and plasmids are well within the level of skill of the art.

As used herein, “nucleic acid” refers to single-stranded and/or double-stranded polynucleotides, such as deoxyribonucleic acid (DNA) and ribonucleic acid (RNA), as well as analogs or derivatives of either RNA or DNA. Also included in the term “nucleic acid” are analogs of nucleic acids such as peptide nucleic acid (PNA), phosphorothioate DNA, and other such analogs and derivatives. When referring to probes or primers, optionally labeled, with a detectable label, such as a fluorescent or radiolabel, single-stranded molecules are contemplated. Such molecules are typically of a length such that they are statistically unique and of low copy number (typically less than 5, preferably less than 3) for probing or priming a library. Generally a probe or primer contains at least 14, 16 or 30 contiguous nucleotides of sequence complementary to or identical to a gene of interest. Probes and primers can be 10, 20, 30, 50, 100 or more nucleotides long.

As used herein, “DNA” is meant to include all types and sizes of DNA molecules including cDNA, plasmids and DNA including modified nucleotides and nucleotide analogs.

As used herein, “nucleotides” include nucleoside mono-, di-, and triphosphates. Nucleotides also include modified-nucleotides, such as, but are not limited to, phosphorothioate nucleotides and deazapurine nucleotides and other nucleotide analogs.

As used herein, “heterologous” or “foreign DNA and RNA” are used interchangeably and refer to DNA or RNA that does not occur naturally as part of the genome in which it is present or which is found in a location or locations and/or in amounts in a genome or cell that differ from that in which it occurs in nature. Heterologous nucleic acid is generally not endogenous to the cell into which it is introduced, but has been obtained from another cell or prepared synthetically. Generally, although not necessarily, such nucleic acid encodes RNA and proteins that are not normally produced by the cell in which it is expressed. Any DNA or RNA that one of skill in the art would recognize or consider as heterologous or foreign to the cell in which it is expressed is herein encompassed by heterologous DNA. Heterologous DNA and RNA may also encode RNA or proteins that mediate or alter expression of endogenous DNA by affecting transcription, translation, or other regulatable biochemical processes.

Examples of heterologous DNA include, but are not limited to, DNA that encodes a gene product or gene product(s) of interest, introduced for purposes of modification of the endogenous genes or for production of an encoded protein. For example, a heterologous or foreign gene may be isolated from a different species than that of the host genome, or alternatively, may be isolated from the host genome but operably linked to one or more regulatory regions which differ from those found in the unaltered, native gene. Other examples of heterologous DNA include, but are not limited to, DNA that encodes traceable marker proteins, such as a protein that confers traits including, but not limited to, herbicide, insect, or disease resistance; traits, including, but not limited to, oil quality or carbohydrate composition. Antibodies that are encoded by heterologous DNA may be secreted or expressed on the surface of the cell in which the heterologous DNA has been introduced.

As used herein, “operative linkage” or “operative association”, or grammatical variations thereof, of heterologous DNA to regulatory and effector sequences of nucleotides, such as promoters, enhancers, transcriptional and translational stop sites, and other signal sequences refers to the relationship between such DNA and such sequences of nucleotides. For example, operative linkage of heterologous DNA to a promoter refers to the physical relationship between the DNA and the promoter such that the transcription of such DNA is initiated from the promoter by an RNA polymerase that specifically recognizes, binds to and transcribes the DNA.

In order to optimize expression and/or in vitro transcription, it may be necessary to remove, add or alter 5′ untranslated portions of the clones to eliminate extra, potential inappropriate alternative translation initiation (i.e., start) codons or other sequences that may interfere with or reduce expression, either at the level of transcription or translation.

Alternatively, consensus ribosome binding sites (see, e.g., Kozak (1991) J. Biol. Chem. 266: 19867-19870) can be inserted immediately 5′ of the start codon and may enhance expression.

As used herein, a sequence complementary to at least a portion of an RNA, with reference to antisense oligonucleotides, means a sequence having sufficient complementarity to be able to hybridize with the RNA, preferably under moderate or high stringency conditions, forming a stable duplex. The ability to hybridize depends on the degree of complementarity and the length of the antisense nucleic acid. The longer the hybridizing nucleic acid, the more base mismatches it can contain and still form a stable duplex (or triplex, as the case may be). One skilled in the art can ascertain a tolerable degree of mismatch by use of standard procedures to determine the melting point of the hybridized complex.

As used herein, “regulatory molecule” refers to a polymer of deoxyribonucleic acid (DNA) or ribonucleic acid (RNA) or a polypeptide that is capable of enhancing or inhibiting expression of a gene.

As used herein, a “promoter”, with respect to a region of DNA, refers to a sequence of DNA that contains a sequence of bases that signals RNA polymerase to associate with the DNA and initiate transcription of RNA (such as pol II for mRNA) from a template strand of the DNA. A promoter thus generally regulates transcription of DNA into mRNA.

As used herein, a “light sensing protein activated promoter” refers to a promotor directly or indirectly activated by light to regulate the transcription. Particular light sensing protein activated promoter provided herein may include NFAT-dependent promoter, PIR3_HSP70 min promoter, C120 promoter, and etc.

As used herein, “expression” refers to the transcription and/or translation of nucleic acid. For example, expression can be the transcription of a gene that may be transcribed into an RNA molecule, such as a messenger RNA (mRNA) molecule. Expression may further include translation of an RNA molecule and translated into peptides, polypeptides, or proteins. If the nucleic acid is derived from genomic DNA, expression may, if an appropriate eukaryotic host cell or organism is selected, include splicing of the mRNA. With respect to an antisense construct, expression may refer to the transcription of the antisense DNA.

As used herein, “transformation/transfection” refers to the process by which nucleic acid is introduced into cells. The terms transfection and transformation refer to the taking up of exogenous nucleic acid, e.g., an expression vector, by a host cell whether or not any coding sequences are in fact expressed. Numerous methods of transfection are known to the ordinarily skilled artisan, for example, by Agrobacterium-mediated transformation, protoplast transformation (including polyethylene glycol (PEG)-mediated transformation, electroporation, protoplast fusion, and microcell fusion), lipid-mediated delivery, liposomes, electroporation, sonoporation, microinjection, particle bombardment and silicon carbide whisker-mediated transformation and combinations thereof (see, e.g., Paszkowski et al. (1984) EMBO J. 3:2717-2722; Potrykus et al. (1985) Mol. Gen. Genet. 199:169-177; Reich et al. (1986) Biotechnology 4:1001-1004; Klein et al. (1987) Nature 327:70-73; U.S. Pat. No. 6,143,949; Paszkowski et al. (1989) in Cell Culture and Somatic Cell Genetics of Plants, Vol. 6, Molecular Biology of Plant Nuclear Genes, eds. Schell, J and Vasil, L. K. Academic Publishers, San Diego, Calif., p. 52-68; and Frame et al. (1994) Plant J. 6:941-948), direct uptake using calcium phosphate (CaPO4; see, e.g., Wigler et al. (1979) Proc. Natl. Acad. Sci. U.S.A. 76:1373-1376), polyethylene glycol (PEG)-mediated DNA uptake, lipofection (see, e.g., Strauss (1996) Meth. Mol. Biol. 54:307-327), microcell fusion (see, EXAMPLES, see, also Lambert (1991) Proc. Natl. Acad. Sci. U.S.A. 88:5907-5911; U.S. Pat. No. 5,396,767, Sawford et al. (1987) Somatic Cell Mol. Genet. 13:279-284; Dhar et al. (1984) Somatic Cell Mol. Genet. 10:547-559; and McNeill-Killary et al. (1995) Meth. Enzymol. 254:133-152), lipid-mediated carrier systems (see, e.g., Teifel et al. (1995) Biotechniques 19:79-80; Albrecht et al. (1996) Ann. Hematol. 72:73-79; Holmen et al. (1995) In Vitro Cell Dev. Biol. Anim. 31:347-351; Remy et al. (1994) Bioconjug. Chem. 5:647-654; Le Bolch et al. (1995) Tetrahedron Lett. 36:6681-6684; Loeffler et al. (1993) Meth. Enzymol. 217:599-618) or other suitable method. Successful transfection is generally recognized by detection of the presence of the heterologous nucleic acid within the transfected cell, such as, for example, any visualization of the heterologous nucleic acid or any indication of the operation of a vector within the host cell.

As used herein, “substantially homologous DNA” or “equivalent DNA” refers to DNA that includes a sequence of nucleotides that is sufficiently similar to another such sequence to form stable hybrids, with each other or a reference sequence, under specified conditions.

As used herein: stringency of hybridization in determining percentage mismatch encompass the following conditions or equivalent conditions thereto:
1) high stringency: 0.1×SSPE or SSC, 0.1% SDS, 65° C.
2) medium stringency: 0.2×SSPE or SSC, 0.1% SDS, 50° C.
3) low stringency: 1.0×SSPE or SSC, 0.1% SDS, 50° C.
   or any combination of salt and temperature and other reagents that result in selection of the same degree of mismatch or matching. Equivalent conditions refer to conditions that select for substantially the same percentage of mismatch in the resulting hybrids. Additions of ingredients, such as formamide, Ficoll, and Denhardt's solution affect parameters such as the temperature under which the hybridization should be conducted and the rate of the reaction. Thus, hybridization in 5×SSC, in 20% formamide at 42° C. is substantially the same as the conditions recited above hybridization under conditions of low stringency. The recipes for SSPE, SSC and Denhardt's and the preparation of deionized formamide are described, for example, in Sambrook et al. (1989) Molecular Cloning, A Laboratory Manual, Cold Spring Harbor Laboratory Press, Chapter 8; see, Sambrook et al., vol. 3, p. B.13, see, also, numerous catalogs that describe commonly used laboratory solutions. It is understood that equivalent stringencies may be achieved using alternative buffers, salts and temperatures. As used herein, all assays and procedures, such as hybridization reactions and antibody-antigen reactions, unless otherwise specified, are conducted under conditions recognized by those of skill in the art as standard conditions.

As used herein, conditions under which DNA molecules form stable hybrids are considered substantially homologous, and a DNA or nucleic acid homolog refers to a nucleic acid that includes a preselected conserved nucleotide sequence, such as a sequence encoding a polypeptide. By the term “substantially homologous” is meant having at least 75%, preferably 80%, preferably at least 90%, most preferably at least 95% homology therewith or a less percentage of homology or identity and conserved biological activity or function.

The terms “homology” and “identity” are often used interchangeably. In this regard, percent homology or identity may be determined, for example, by comparing sequence information using a GAP computer program. The GAP program utilizes the alignment method of Needleman and Wunsch (J. Mol. Biol. 48:443 (1970), as revised by Smith and Waterman (Adv. Appl. Math. 2:482 (1981). Briefly, the GAP program defines similarity as the number of aligned symbols (i.e., nucleotides or amino acids) which are similar, divided by the total number of symbols in the shorter of the two sequences. The preferred default parameters for the GAP program may include: (1) a unary comparison matrix (containing a value of 1 for identities and 0 for non-identities) and the weighted comparison matrix of Gribskov and Burgess, Nuc. Acids Res. 14:6745 (1986), as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

By sequence identity, the number of conserved amino acids are determined by standard alignment algorithms programs, and are used with default gap penalties established by each supplier. Substantially homologous nucleic acid molecules would hybridize typically at moderate stringency or at high stringency all along the length of the nucleic acid of interest. Preferably the two molecules will hybridize under conditions of high stringency. Also contemplated are nucleic acid molecules that contain degenerate codons in place of codons in the hybridizing nucleic acid molecule.

Whether any two nucleic acid molecules have nucleotide sequences that are at least 75%, 80%, 85%, 90%, 95%, 96%, 97%, 98% or 99% “identical” can be determined using known computer algorithms such as the “FAST A” program, using for example, the default parameters as in Pearson and Lipman, Proc. Natl. Acad. Sci. USA 85:2444 (1988). Alternatively the BLAST function of the National Center for Biotechnology Information database may be used to determine relative sequence identity.

In general, sequences are aligned so that the highest order match is obtained. “Identity” per se has an art-recognized meaning and can be calculated using published techniques. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptide sequences, the term “identity” is well known to skilled artisans (Carillo, H. & Lipton, D., SIAM J Applied Math 48:1073 (1988)). Methods commonly employed to determine identity or similarity between two sequences include, but are not limited to, those disclosed in Guide to Huge Computers, Martin J. Bishop, ed., Academic Press, San Diego, 1994, and Carillo, H. & Lipton, D., SIAM J Applied Math 48:1073 (1988). Methods to determine identity and similarity are codified in computer programs. Preferred computer program methods to determine identity and similarity between two sequences include, but are not limited to, GCG program package (Devereux, J., et al., Nucleic Acids Research 12(I):387 (1984)), BLASTP, BLASTN, FASTA (Atschul, S. F., et al., J Molec Biol 215:403 (1990)).

Therefore, as used herein, the term “identity” represents a comparison between a test and a reference polypeptide or polynucleotide.

For example, a test polypeptide may be defined as any polypeptide that is 90% or more identical to a reference polypeptide.

As used herein, the term at least “90% identical to” refers to percent identities from 90 to 99.99 relative to the reference polypeptides. Identity at a level of 90% or more is indicative of the fact that, assuming for exemplification purposes a test and reference polynucleotide length of 100 amino acids are compared. No more than 10% (i.e., 10 out of 100) amino acids in the test polypeptide differs from that of the reference polypeptides. Similar comparisons may be made between a test and reference polynucleotides. Such differences may be represented as point mutations randomly distributed over the entire length of an amino acid sequence or they may be clustered in one or more locations of varying length up to the maximum allowable, e.g., 10/100 amino acid difference (approximately 90% identity). Differences are defined as nucleic acid or amino acid substitutions, or deletions.

As used herein, “conservative amino acid substitutions”, such as those set forth in Table 1, are those that do not eliminate biological activity. Suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Bejacmin/Cummings Pub. co., p. 224). Conservative amino acid substitutions are made, for example, in accordance with those set forth in TABLE 1 as follows:

TABLE 1

| Original residue | Conservative substitution |
|---|---|
| Ala (A) | Gly; Ser, Abu |
| Arg (R) | Lys, orn |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val; Met; Nle; Nva |
| Leu (L) | Ile; Val; Met; Nle; Nva |
| Lys (K) | Arg; Gln; Glu |
| Met (M) | Leu; Tyr; Ile; NLe Val |
| Ornithine | Lys; Arg |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu; Met; Nle; Nva |

Other substitutions also are permissible and may be determined empirically or in accord with known conservative substitutions.

As used herein, the term “equivalent protein” or “equivalent peptide” means that the two proteins or peptides have substantially the same amino acid sequence with only amino acid substitutions (such as, but not limited to, conservative changes) or structure and the any changes do not substantially alter the activity or function of the protein or peptide. When equivalent refers to a property, the property does not need to be present to the same extent (e.g., two peptides can exhibit different rates of the same type of enzymatic activity), but the activities are usually substantially the same.

As used herein, the term “light sensing protein”, “light sensitive protein”, “light activated protein”, “protein photoreceptor” refer to proteins which react to light via photoisomerization or photoreduction, thus initiating changes of the proteins which trigger a functional effect upon irradiation with light of a selected wavelength. “Light sensing protein”, “light sensitive protein”, “light activated protein”, and “protein photoreceptor” can include: melanopsin, phytochrome, cytochrome, photopsin, rhodopsin, protein kinase C, PhyB/PIF3 & PhyB/PIF6, EL222, SOUL, CarH, LOV (light oxygen voltage sensing domain), PYP (photoactive yellow protein), OPN5, UVR8, cryptochrome, and phototropin, or any mutant variants or fragments of the aforementioned proteins having substantially the same function as to the wild type proteins.

As used herein, the term “GLP-1” or “GLP-1 molecules” refers to GLP-1 proteins, peptides, polypeptides, analogs, mimetics, derivatives, isoforms, fragments and the like which retain at least one biological activity of native GLP-1.

As used herein, the term “EGFP” refers to an enhanced green fluorescence protein, peptides, polypeptides, analogs, mimetics, derivatives, isoforms, fragments and the like which retain at least one biological activity of native EGFP.

As used herein, “IRES” refers to a region of a nucleic acid molecule, such as an mRNA molecule, that allows internal ribosome entry sufficient to initiate translation, which initiation can be detected in an assay for cap-independent translation (see, e.g., U.S. Pat. No. 6,171,821). The presence of an IRES within an mRNA molecule allows cap-independent translation of a linked protein-encoding sequence that otherwise would not be translated.

Internal ribosome entry site (IRES) elements were first identified in picornaviruses, which elements are considered the paradigm for cap-independent translation. The 5′ UTRs of all picornaviruses are long and mediate translational initiation by directly recruiting and binding ribosomes, thereby circumventing the initial cap-binding step. IRES elements are frequently found in viral mRNA, they are rare in non-viral mRNA. Among non-viral mRNA molecules that contain functional IRES elements in their respective 5′ UTRs are those encoding immunoglobulin heavy chain binding protein (BiP) (Macejak et al. (1991) Nature 353:90-94); Drosophila antennapedia (Oh et al. (1992) Genes Dev, 6:1643-1653); D. Ultrabithorax (Ye et al. (1997) Mol. Cell Biol. 17:1714-21); fibroblast growth factor 2 (Vagner et al. (1995) Mol. Cell Biol. 15:35-44); initiation factor elF4G (Gan et al. (1998) J. Biol. Chem. 273:5006-5012); proto-oncogene c-myc (Nanbru et al. (1995) J. Biol. Chem. 272:32061-32066; Stoneley (1998) Oncogene 16:423-428); IRES_{H}; from the 5′UTR of NRF1 gene (Oumard et al. (2000) Mol. and Cell Biol., 20(8):2755-2759); and vascular endothelial growth factor (VEGF) (Stein et al. (1998) Mol. Cell Biol. 18:3112-9).

Present system described herein features genetically engineered active cells (“engineered cells”) that produce or are capable of producing one or more therapeutic agents. The therapeutic agent may be a biological substance, such as a nucleic acid (e.g., a nucleotide, DNA, or RNA), a polypeptide, a lipid, a sugar (e.g., a monosaccharide, disaccharide, oligosaccharide, or polysaccharide), a small molecule, etc. In some embodiments, the therapeutic agent is a polypeptide. Each engineered cell comprises a promoter operably linked to a nucleotide sequence encoding the polypeptide. In such an implementation, the promoter can essentially be a nucleotide sequence. In some embodiments, the therapeutic agent is a replacement therapy or a replacement protein, e.g., useful for the treatment of a blood clotting disorder or a lysosomal storage disease in a subject. The engineered cells are housed in an implantable device

In some embodiments, an implantable device contains the engineered cells, which can be provided as a cluster or disposed in a microcarrier. In some embodiments, the engineered cells produce or release a therapeutic agent (e.g., a polypeptide) for at least 0.5 day, 1 day, 10 days, or more, when the implantable device implanted into a subject. In one embodiment, more than one therapeutic agent are produced by the engineered cells. In one embodiment, the implantable device may include one or more types of engineered cells, one type of the engineered cells may produce a therapeutic agent which is different from the therapeutic agents produced by other types of the engineered cells.

Cells suitable for encapsulation and transplantation are generally secretory or metabolic cells (i.e., they secrete a therapeutic factor or metabolize toxins, or both) or structural cells (e.g., skin, muscle, blood vessel), or metabolic cells (i.e., they metabolize toxic substances). In some embodiments, the cells are naturally secretory, such as islet cells that naturally secrete insulin, or naturally metabolic, such as hepatocytes that naturally detoxify and secrete. In some embodiments, the cells are genetically engineered to express a recombinant protein, such as a secreted protein or metabolic enzyme. Depending on the cell type, the cells may be organized as single cells, cell aggregates, spheroids, or even natural or bioengineered tissue. In some embodiments, the particle comprises an epithelial cell, endothelial cell, fibroblast cell, mesenchymal stem cell, keratinocyte cell or an islet cell or a cell derived from any of the foregoing cell types. In some embodiments, the particle comprises a retinal pigment epithelial (RPE cell) or a mesenchymal stem cell (MSC). In some embodiments, the particle comprises an engineered cell (e.g., an engineered RPE cell or an engineered MSC). In some embodiments, the disclosed compositions contain cells genetically engineered to produce a therapeutic protein or nucleic acid. In these embodiments, the cell can be a stem cell (e.g., pluripotent), a progenitor cell (e.g., multipotent or oligopotent), or a terminally differentiated cell (i.e., unipotent). The cell can be engineered to contain a nucleic acid encoding a therapeutic polynucleotide such miRNA or RNAi or a polynucleotide encoding a protein. The nucleic acid can be integrated into the cells genomic DNA for stable expression or can be in an expression vector (e.g., plasmid DNA). The therapeutic polynucleotide or protein can be selected based on the disease to be treated and the site of transplantation. In some embodiments, the therapeutic polynucleotide or protein is anti-neoplastic. In other embodiments, the therapeutic polynucleotide or protein is a hormone, growth factor, or enzyme.

In another aspect, the present disclosure features a method of treating a subject comprising administering to the subject an implantable device housing the engineered cells producing at least one therapeutic agents.

In one illustrative embodiment, the subject is a human and the engineered active cell is a human cell. Alternatively, the subject may be a dog, cat, or other animal. In some embodiments, the therapeutic agent produced by the engineered cell(s) is a replacement therapy or a replacement protein, e.g., useful for the treatment of metabolic diseases. In some embodiments, the implantable device is formulated for implantation or injection into a subject.

The produced therapeutic agents can be evaluated by an art-recognized reference method, e.g., polymerase chain reaction or in situ hybridization for nucleic acids; mass spectroscopy for lipid, sugar and small molecules; microscopy and other imaging techniques for agents modified with a fluorescent or luminescent tag, and ELISA or Western blotting for polypeptides. In some embodiments, the implantable device comprises an encapsulating component (e.g., formed in situ on or surrounding the engineered cells, or preformed prior to combination with the engineered cells). In other embodiments, the implantable device is chemically modified, as described herein.

Thus, described herein is a hybrid bioelectronics/engineered cells pharmacy that enables the production of therapeutic agents within the subject. The therapeutic agents can be used to control pain, treat metabolic disorders, treat immune system disorders, treat psychiatric disorders, improve fertility, and any other medical or health conditions requiring a frequent and/or precise administration of therapeutic agents.

The present invention provides a therapy having a timing and dosing control which far exceeds the existing therapies and/or bioelectronics. The system is able to achieve 1) specific biological action on select target receptors or molecules that cannot be accomplished with current bioelectronics, and 2) precise control of timing and dosage that cannot be accomplished with current synthetic biology.

In this embodiment, the living pharmacy includes engineered cells that produce therapeutic peptides with a timing and dose profile that is tightly controlled by optical triggers from an implanted bioelectronic carrier device (i.e., implant device). Together with a subcutaneous bioelectronic carrier, the system overcomes the major challenges facing hybrid bioelectronic devices, including: 1) selective activity on biological targets, 2) precise control of biomolecule production, 3) high dose to load volume ratio, 4) protection from the host's immune response, and 5) wireless data and power transfer through biological tissue. In alternative implementations, the system may have fewer, additional, and/or different features.

The biohybrid system of the present invention provides a general platform for precise drug delivery and regulation that can be implanted long-term to treat short term or long term diseases, physical and/or mental health conditions, as well as improve user health and performance, without the need to carry pharmaceuticals. In one embodiment, the system minimizes the adverse health consequences of circadian misalignment by achieving at least a 50% reduction in entrainment time using an implanted living biohybrid pharmacy that remains functional for an extended period of time (e.g., 30 days, 60 days, 90 days, etc.). In other embodiments, the disclosed invention provides treatments to diseases and/or physiological conditions including metabolic diseases, e.g., obesity and diabetes (e.g., Type 1, Type 2) by producing metabolically active molecules, e.g., leptin, ACTH, insulin, and GLP-1; cancers by producing therapeutic cytokines e.g., IL-2, IL-12, IL-15, GCSF; autoimmune diseases by producing regulated molecules e.g., IL-10, IL-35, treatment resistant depression and pains by producing neuropeptides e.g., GLYX-13, rapastinel, and ziconotide; osteoporosis by producing PTH; infertility by producing gonadotropin releasing hormone GnRH; and etc. To achieve these metrics, the system focuses on five main innovations to overcome barriers of current bioelectronic and synthetic biology technologies, as well as an innovative approach to accelerating entrainment. These innovations, which are described in detail below, include performing selective activity on biological targets using natural peptides, precisely controlling biomolecule production, obtaining a high dose to load volume ratio, providing protection from the host's immune response, and wirelessly transmitting data and power through biological tissue.

With respect to selective activity on biological targets using natural peptides, the inventors have proposed using engineered allogeneic cells to produce select peptides that are otherwise naturally produced by the body to control pain, fight disease, regulate sleep cycles, treat metabolic related conditions, and etc. It is to be understood that in other applications the system can be used to produce other types of therapeutic agents. The body naturally produces these native peptides which are structurally similar to their recombinant counterparts. However, the native peptides diverge in potency and bioactivity. Significantly, it is noted that native peptides have not been commercialized due to their instability. However, the inventors have determined that a cell delivery platform which supports on demand in situ production use of native peptides as therapeutics is feasible. These naturally produced molecules are excellent candidates to regulate the specific activity of central and peripheral circadian clocks because they act selectively on these biological targets and do not elicit the immune responses that are shortcomings of recombinant peptides or exogenous drugs (i.e., antidrug antibodies). Furthermore, these peptides feature short metabolic half-lives, and are useful for relatively fast cessation of drug production. These traits make the proposed cell platform uniquely suited to deliver such biologics on demand. Additionally, it has been demonstrated that allogeneic cells encapsulated and implanted in vivo can survive for greater than 130 days in non-human primates without immunosuppression, suggesting that the proposed solution can enable living engineered cells-based devices with lifetimes that can extend for several months, or even years.

The system is also able to perform precision dosing with closed-loop bioelectronic control. A key challenge for biological production of therapeutic agents is controlling the production levels, which can vary due to cell health, temperature, and metabolism. To overcome this challenge, the system includes a state-of-the art bioelectronic feedback control system based on optogenetically controlled therapy production and fluorescent tracking of therapy production levels. In alternative embodiments, the feedback control system may not be used. Cells engineered with optogenetic systems start protein production in response to exposure to specific activating light signals. By controlling light exposure, production of therapeutic agents can be controlled.

Another innovation of the system is a bioelectronic feedback loop based on fluorescent tracking of the production levels. To create this feedback control loop, the cells are engineered to produce a fluorescent protein at a fixed ratio relative to the therapeutic agents. Using this fluorescence measurement as the feedback signal, the system is able to regulate the on time of the engineered cells to maintain a stable fixed point of therapeutic agents production with precision that exceeds synthetic biological feedback loops. In some embodiments, in addition to the fluorescent signal provided by the fluorescent proteins, the bioelectronic feedback loop is based on biochemical signals which can be electronically detected. In addition to the fluorescent signal, the biochemical signals may include bioluminescence signal, impedance signal, pigment signal, and free radical signal.

In some embodiments, the system also provides high-dose to load volume with on-chip life support and engineered cells. To support a higher concentration of therapeutic agents produced by the implanted device, one could increase the density of the engineered cells inside the chassis. However, the maximum cell density is currently limited by the amount of diffuse oxygen available in the subcutaneous space. To reach higher cell densities, the carrier is engineered to produce local O₂ with the bioelectronic carrier. Furthermore, using synthetic biology tools, the system amplifies transcription of the therapeutic peptides and programs cells to be resilient to senescence and cell death.

In another aspect of the invention, the system also provides protection from the host immune response using a small molecule coating. Specifically, engineered cells are encapsulated within a life support system that protects them from the immune system of the host and that supports cell viability and productivity. Hydrogels and permeable or semi-permeable membrane biomaterials can be used to block cells from the body's immune system via their physical, hierarchical pore structure and biochemical functionalization. The hydrogels and permeable or semi-permeable membrane biomaterials further promote vascularization near the device/tissue interface to boost oxygenation from the body's circulatory system.

In some embodiments, the system also performs efficient wireless data and power transfer through tissue using magnetoelectrics. Traditional wireless power delivery by electromagnetic or ultrasound waves has to overcome absorption by tissue and impedance mismatches between air, bone, and tissue, and such techniques often struggle to provide large powers to small bioelectronic devices. In contrast, magnetic fields are not affected by tissue-absorption or differences in interfacial impedances. The proposed approach uses recently-developed magnetoelectric (ME) technology and custom low power application specific integrated circuits (ASICs) to enable compact, reliable, wireless transmission of power and data, providing superior power densities and alignment tolerance. This approach for wireless power can enable ultra-miniature versions of the implantable device that can be injectable. Alternatively, the system can use other sources of power and data transfer such as inductive coupling, photovoltaic data and power control, radio frequency (RF) data and power control, inductive data and power control, ultrasound data and power control, direct current (DC) coupled data and power control, etc. In alternative implementations, battery power or energy harvesting from the body could reduce or eliminate the need for wireless power.

The system can be used for delivery of single or multiple therapeutics. In one embodiment, the engineered cells produce one or more therapeutic agents. In another embodiment, there are more than one type of engineered cells housed either in one cell housing, or each type of the engineered cells is housed in separated housings. In one exemplary embodiment of multiple therapeutic delivery, multi-clock targeting with precision timing for circadian rhythm regulation can be performed by the system, as discussed below.

Unlike bioelectronic or gated biofluidic systems that feature pre-filled (or even refillable) reservoirs of drugs, the system delivers naturally-occurring peptides throughout its functional lifetime without the need to stock, carry, or refill therapies that are vulnerable to loss, degradation, or that add to the already burdensome load carried by the user. The developed technology will serve as a platform whereby the optical control and feedback to achieve precision therapies can be applied to delivery of a broad swath of naturally occurring peptides/proteins by following the procedures and protocols described herein.

Thus, the disclosed system provides a hybrid bioelectronics platform and forms the basis and components for a number of bioelectronic and biohybrid tools to address or alleviate dysfunction and injury, to enhance readiness and performance, to treat pain, to treat disease, improves metabolism, and etc. The rationale behind the system, along with details of its implementation, use, and testing are described in more detail below.

### Biohybrid System for on Demand Therapy

Engineering a cell-based hybrid bioelectronic system for on demand therapy is a challenge involving a careful balance between cellular and bioelectronic device engineering. Several factors have to be taken into consideration to balance the strengths of each pillar and to minimize their drawbacks and deficiencies. Cells possess their own natural machinery to synthesize and release specific biomolecules. Furthermore, their machinery can be hacked to externally induce such production, but without the level of timing, control, and user interfacing that is possible with bioelectronics. On the other hand, optoelectronic components have inherent chemical and mechanical mismatches with cells and tissue that can limit their lifetime (degradation, rejection), or make them otherwise incompatible with reliable use in vivo. Striking the proper balance between the two is important.

Key design decisions that permeate the system focus on (i) promoting long-term viability/efficiency and (ii) controlling and creating feedback-loop of therapeutic agents production. To promote viability of engineered cells, the system both genetically engineers cells to be more resilient, and in some embodiments can also produce O₂ to support them. For control and feedback-loop, optical induction can be used. Compared to other cellular control mechanisms, optical induction methods enable fast response, tunable, localized induction properties (wavelength control), and are readily integrated into the platform with minimal power and size demands. Furthermore, optoelectronic cell interfacing enables innovative precision low-power dosing control.

### Technological Components of the System

In an illustrative embodiment, the system includes an implantable device, (e.g., subcutaneous implanted) featuring individually-controlled cell housing, and an external wearable hub (extHub) (hardware and software) for power, user interface, and sensing. In alternative implementations, only the implantable device may be used. It should be noted that, in the application, the term “cell house”, “cell housing” and “cell well” are used interchangeably.

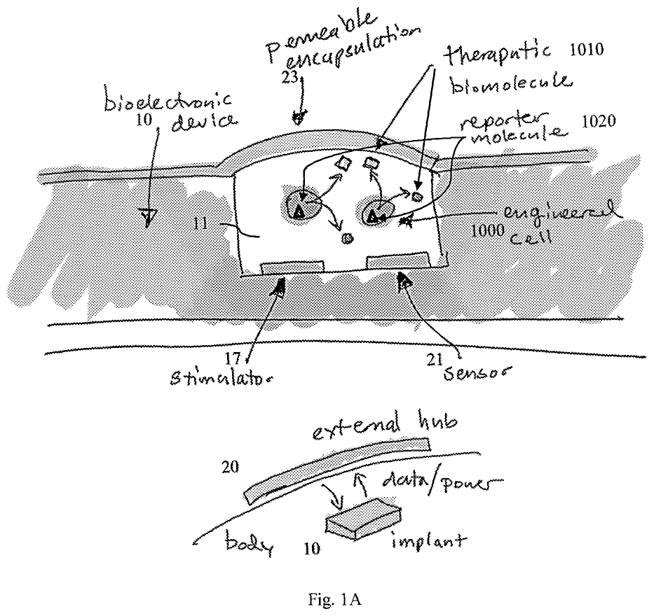

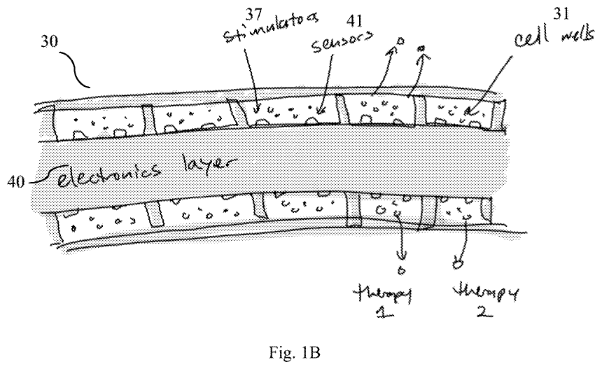

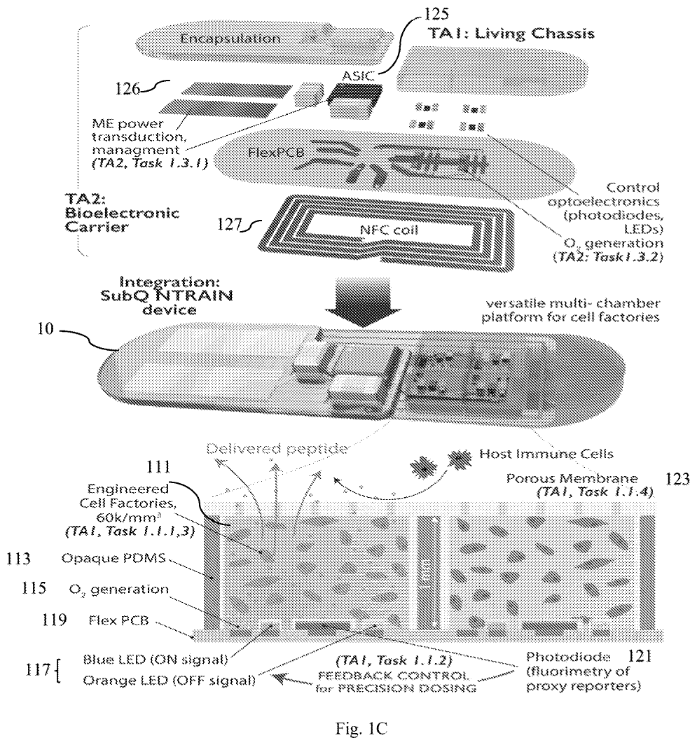

FIG. 1A provides an illustrative diagram showing the general structure of a unit of the bioelectronics device. As shown in the lower panel, the bioelectronics may comprising a unit of the implantable device 10 inside the body of a user, and an external hub 20 located outside the body of the user. The external hub 20 is in communication with the implantable device 10 for power charging and data exchanging/transmission. As shown in the upper panel, the implantable device 10 comprising a cell housing 11 for containing engineered cells 1000 which produces the therapeutic agents 1010 and a reporter agent/molecule 1020. A stimulator 17 for triggering the production of the therapeutic agents 1010 and the reporter agent/molecule 1020 by the engineered cells 1000 locates in the cell housing 11. A sensor 21 for sensing the reporter agent/molecule 1020 also locates in the cell housing 11. Both the stimulator and the sensor locate in vicinity to the engineered cells 1000 such that they effectively stimulate the production of the therapeutic agents 1010 and the reporter agent/molecule 1020 and sensing the production of the reporter agent/molecule 1020. At least one side of the implantable device 10 are coated or encapsulated with a permeable material/membrane 23 which shield the implantable device 10 from the immune system/cells of the user.

FIG. 1B provides an illustration diagram of another embodiment of the implantable device 30 which has multiple cell wells/housings 31. The implantable device has more than one cell well/housing 31 attached to an electronic layers 40. Each of the cell well/housing 31 has an individual stimulator 37 and an individual sensor 41. In one embodiment, the stimulators 37 and sensor 41 locate in vicinity to the engineered cells 1000 such that they effectively stimulate the production of the therapeutic agents 1010 and the reporter agent/molecule 1020 and sensing the production of the reporter agent/molecule 1020. In one embodiment, cell wells/housings 31 contain engineered cells producing same therapeutic agent 1010. In another embodiment, different types of engineered cells 1000 producing different therapeutic agents 1010 may be each contained in a separate cell well/housing 31, such that the each type of engineered cells may be individually controlled by the stimulator and the sensor in each cell well/housing 31, so as to produce a particular therapeutic agents 1010 for a specific amount and/or at a specific time different from that of in the other cells/housings.

FIG. 1C depicts an alternative embodiment showing a subcutaneous NTRAIN device 110, including a cross-section that depicts method of operation and associated tasks for engineered components in accordance with an illustrative embodiment. The implanted subcutaneous device includes (i) genetically engineered allogeneic mammalian cells programmed to deliver peptide therapeutics in accordance with an optical trigger 117, (ii) hybrid synthetic biology/bioelectronic feedback control to provide precision dosing 121, (iii) O₂ generation capabilities/device 115, (iv) immune-isolating materials for enhanced cell viability and protection 123, (v) a custom application-specific integrated circuit (ASIC) 125 for low-power feedback control, temperature sensing, and power management, (vi) mm-scale magnetoelectric transducers 126 for wireless power and data/controls downlink, and (vii) a near field communication (NFC) coil 127 for wireless data uplink. In alternative implementations, the device can have fewer, additional, and/or different features.

In one embodiment, the implanted device is approximately 0.8 cm×3 cm, with a thickness of about 2-3 mm, and bendable over a 1 centimeter radius of curvature. In alternative embodiments, different dimensions and/or radius of curvature may be used. Each cell well/housing 111 can include one or more isolated compartments (or enclosures). In one embodiment, each cell well/housing houses about 240k cells, 2×2×1 mm in size. Alternatively, a different number of cells and/or a different compartment size may be used. At the base of the compartment is a bioelectronic carrier, on which control LEDs (stimulator) 117 initiate and stop peptide production. Specifically, an LED/photodiode pair (sensor) 121 is used to probe production of destabilized fluorescent proteins (e.g., GFP or EGFP) which are produced as a proxy for the delivered peptide, providing optical feedback of production levels, for closed loop dosage control. The compartment also contains O₂ generating particles or an O₂ generating electrochemical device 115 in one embodiment, which allows the system to have increased density of engineered cells within the chassis. In an illustrative embodiment, the housings that form the cell compartments can be made opaque by using opaque PDMS walls 113 between the compartments 111 to minimize crosstalk of the optical control signals between cell compartments.

In one embodiment, the implantable device can be implanted subcutaneously, pericardially, intracranially, or intraperitoneally for delivery of the therapeutic agents, so as to customized to the subject's needs. In one embodiment, the implantable device can be implanted in a proper location for delivering the therapeutic agents either locally or systematically.

### Implementation and Operation: Implantation, Implementation, and Life Cycle

In an illustrative embodiment, implementation of the system involves implantation of the subcutaneous device in a subject. The subcutaneous device can be implanted via an outpatient procedure at approximately 2 cm or less below the skin in the abdomen. This implantation location can vary, and depends on the balance of comfort/adoption and systemic delivery efficacy. Additionally, in alternative embodiments, a different implantation location may be used such as omentum, fat, muscle, brain, heart, skin, hips, joints, etc. During use, the implanted device can be secured in a subcutaneous pocket to prevent movement. In some embodiments, the user is outfitted with an external hub in a harness and provided startup operation instructions via an application running on a user device in one embodiment. These instructions guide the user in how to place the external hub by monitoring a power coupling between the implant and the external hub.

In an ideal use case, the subcutaneous device can be implanted for a needed duration of time (e.g., length of a deployment, length of a project or job, etc.) and explanted via outpatient procedure once the duration of time ends. Depending on the materials used and the implementation, the system can have a 60 day lifetime, a 130 day lifetime, a lifetime measured in years, etc. For example, it is anticipated that, using the technology described herein, the system could last for years and that repeated administration would be possible. In one embodiment, the engineered cells can be developed to include a genetically inducible safety kill switch to ensure that the cell therapy can be terminated should there be an untoward event during patient use. In the event that the device needs to be rendered non-functional, kill switch activation is initiated by an FDA-approved small molecule biologic. In an illustrative embodiment, viability of cells can be tracked optically to confirm efficacy of the kill switch.

### Engineered Cells and Optogenetic System

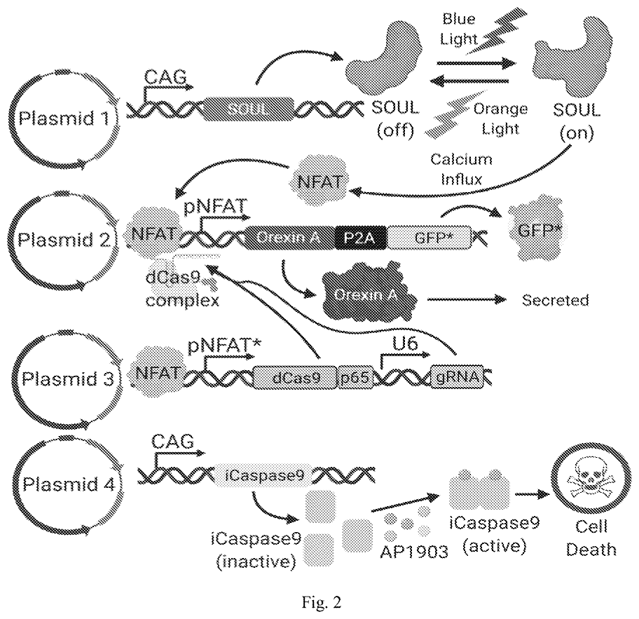

As shown in FIG. 2 reflecting an illustrative embodiment of a system used for circadian rhythm control, RPE cells are engineered to produce high levels of the desired therapeutic proteins (e.g., GLP-1 and Orexin A) on an optical trigger. In one embodiment, a melanopsin based optogenetic system can be used. In other embodiments a step-function opsin or dimerizable transcription factor (e.g., EL222) or split transcription factor (e.g., PhyB-TAD, DBD-PIF6) can be used. Additionally, the cells can be engineered to co-express a fluorescent reporter protein, for example, a destabilized GFP (GFP*) in a fixed ratio with GLP-1 and Orexin A, such as 1:1, allowing the system to observe the expression of GLP-1 and Orexin A in real time by using the easily readable destabilized GFP* fluorescence as a proxy. Additionally, a small-molecule-inducible kill switch can be engineered into the cells to allow for easy termination of the cells, rendering the device inactive. FIG. 2 is a graphical depiction of the proposed synthetic biology circuit for optogenetic control of the peptide therapeutic Orexin A in accordance with an illustrative embodiment. Preliminary data demonstrates the utility and feasibility of this architecture.

Each of the engineered cells have an optogenetic system. Using engineered cells enables the use of an optogenetic control system to control and produce the desired therapies. Using optogenetic systems, dosing can be controlled by modulating the amount of time that the cells are in the on state. Cells are activated to the “ON” state by exposure to light from LEDs of the stimulating system housed within the bioelectric device. Cells in this “ON” state actively transcribe the therapeutic agents needed to produce the therapeutic.

### Achieving Enhanced Dosing for Therapy

In order to engineer the cells to reach therapeutic dosing, and produce higher quantities of GLP-1 and Orexin A, a catalytically dead version of a CRISPR/Cas9 system (termed dCas9) can be used. The dCas9 system binds to a DNA site-specifically, but does not make any cuts or double-strand breaks. In the illustrative embodiment, the dCas9 can be deployed to recruit transcription activation domains to inserted copies of the NFAT promoter. This will allow amplification of the therapeutic protein and GFP* in a stoichiometrically equal manner amenable to high throughput screening of activation levels and quantification of kinetics. Using this system enables target-agnostic gene activation in a highly specific manner and provides a toolbox of validated synthetic biology tools to tailor activation and kinetics to ideal levels, such as synthetic promoters (NFAT or others), protein degradation tags, and 3′UTR variants among others to facilitate gene amplification only when desired.

### Fluorescent Reporter

Since some embodiments of system utilizes the destabilized GFP (GFP*), co-expressed with the therapeutic, one can observe the production of the therapeutic in real time by observing the fluorescence from the GFP*. Since this protein has a half-life of approximately 7 minutes, it provides an accurate real-time indicator of production levels. This real-time observation of protein production enables feedback for precision dosing control, which can be quantified. In some embodiments, in addition to the destabilized GFP, other biochemical agents which produce signals that can be electronically detected may be used as the reporter. In addition to the fluorescent proteins, the reporter may be one of biochemical agents producing bioluminescence signal, impedance signal, pigment signal, or free radical signal.

### Resilience to Apoptosis

In an illustrative embodiment, the engineered cells are designed to be durable to apoptosis and senescence, which is important for prolonged and durable expression over the course of usage. To do this, parallel genetic screening is conducted to find genetic modifications that confer resistance to apoptosis and senescence, but that retain the ability for robust kill switch operation. By applying a selective pressure that elicits these phenotypes in the engineered RPE cells and then sequencing them, the system will enrich for cells harboring genotypes robust to these conditions. These genotypes are then recapitulated in an engineered cell line to be encapsulated as a living drug factory.

### Kill Switch

An important consideration with cell-based therapeutics is that the body may reject the cells, leading to a harmful immune response. Additionally, the user may want to render the system inactive. To address this issue, a kill switch is engineered into the cells. Because it has been used in multiple clinical trials and has shown to be safe, the small molecule inducible kill switch iCaspase 9 (iCasp9) can be used in one embodiment. This will allow for the controlled apoptosis of the implanted cells by administering the small molecule AP1903. The molecule can be administered orally or intravenously in some embodiments. Alternatively, in in one embodiment, the system can feature a small on-board payload of the molecule to be released electronically. In other alternative embodiments, a different type of kill switch may be used.

As shown in FIG. 2, in an illustrative embodiment, plasmids are designed for therapeutic protein expression. In one embodiment, 4 plasmids can be used as follows: plasmid (1), codes for a optogenetic system driven by a CAG promoter having SEQ ID No: 1 or its equivalent, to enable constitutive expression of the optogentic system, e.g., production of opsin SOUL; plasmid (2) codes for therapeutic protein (i.e., GLP-1 or Orexin A) linked with GFP* via a linker such as P2A, all driven by pNFAT (activated by NFAT) having a SEQ ID No: 2, plasmid (3) codes for dCas9 modification of protein expression levels and can include a unique pNFAT driving transcription of a dCas9 coding region fused to copies of the transcription activation domains p65 or HSF1, or to the human p300 acetyltransferase (p300), plasmid (4) codes for iCaspase 9 being driven by a CAG promoter. Any of plasmids (1)-(4) may have a backbone of SEQ ID No: 29.

The components of plasmid (3) can be non-virally-derived domains found in human proteins that activate gene expression and will be modulated in copy number to elicit desired amounts of expression. Downstream of plasmid (4) is a synthetic 3′UTR and a U6 promoter driving transcription of the gRNA to target the therapeutic gene promoter for activation. Each plasmid can have a different selection marker (e.g., puromycin, neomycin, blasticidin, and zeocin) and be engineered to have the backbone to allow for lipofectamine transfection with PiggyBac transposase genomic integration.

For cell engineering, in one embodiment, an allogenic human cell line, ARPE-19 (retinal pigment epithelium, or RPE), was chosen because it is non-tumorigenic, displays contact inhibited growth characteristics, is amenable to genetic modification, and has been shown safe in previous human trials. Genetic components can be introduced using the standard piggyBac transposase system to the engineered RPE cells. Other transfection method commonly known in the art can also be used.

In vitro validation and optimization is also performed via fluorescence output and kinetics. For example, the system can measure GFP* after stimulation by blue light and orange light via a live-cell plate reader over the duration of expression. Using this as the basis for further engineering, expression is tuned to be stronger by modifying the dCas9 system as follows: 1) adding more copies of transactivation domains; 2) using stronger activators (e.g., p300); 3) adding more NFAT binding sites to the promoter region; 4) and/or tuning the Kozak sequence. In one embodiment, synthetic 3′UTR variants and degradation tags are used to control stability of the mRNA transcript and protein, respectively.

In vitro validation and optimization of therapeutic outputs is also performed. Therapeutic outputs can be monitored via qPCR, RNA-seq, ELISA, and Western blot across fixed intervals following stimulation by varying durations of blue light and orange light. GFP* production can also be determined via fluorescence reading and compared to GLP-1 and Orexin A production by way of ELISA measurements to confirm a 1:1 stoichiometric ratio. Small molecule kill switch validation can also be performed. To show that the kill switch functions as expected, cells can be cultured with AP1903 (the trigger molecule), and cell viability can be assayed via live-dead staining at various time points after culturing. To determine apoptotic and senescence resistance, the system can also screen for senescence and apoptosis resistant cells using CRISPR guide RNA (gRNA) knockout libraries in combination with doxorubicin, cisplatin, and/or DMSO challenge for a total of 4 different screens (using DMSO as a control). Cells harboring resistance genotyped and iCaspase9 are administered to ensure that the kill switch retains function. Cell fitness, proliferation, viability, and expression levels can be validated through morphological evaluation, BrdU incorporation, MTT assay, and ELISA, respectively.

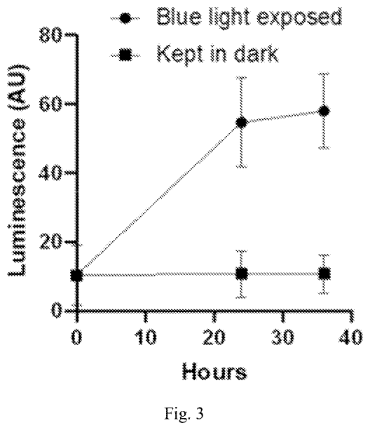

In alternative embodiments, an optogenetic system other than the above-discussed systems to perform cell activation may be used. Other optogenetic system that can be used include melanopsin, EL222 and PhyB/PIF6, which, while they do not have the trigger benefit, but are more established and are shown to work in multiple situations. FIG. 3 depicts preliminary data showing that ARPE-19 cells can be made to express luciferase with high on/off ratio in response to blue light using an EL222 optogenetic system in accordance with an illustrative embodiment.

### Precision Control of Dosing Based on Optical Feedback

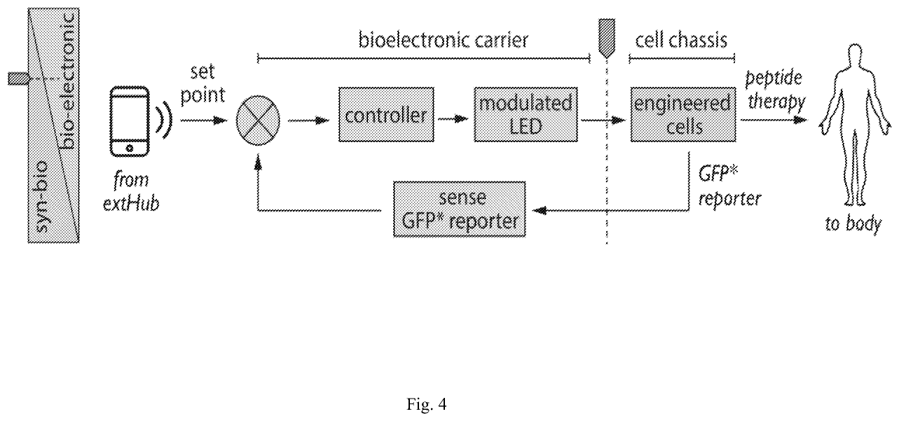

To enable precise and controllable drug production levels despite changes in cell health, stress, and metabolism, a hybrid bioelectronic feedback control system can be created and used. This control system exploits synthetic biology to produce bioactive peptide therapies, and a bioelectronic layer for precise feedback control of production levels. FIG. 4 shows a biohybrid precision control scheme based on co-production of therapeutic peptide and proxy reporter fluorophore (for example, GFP*) in accordance with an illustrative embodiment. As shown, optoelectronics such as photodiode are used to sense and adjust optical stimulation periods to maintain a given setpoint for delivery of therapeutic agents.

To implement this hybrid feedback control system, light source of stimulation system (LEDs) can be integrated into the implantable device to drive optogenetic channels which regulate therapeutic agents production in the engineered cells. To provide this control signal with minimal power consumption, step-function opsins that are activated and inactivated by different color LEDs are used. Specifically, below each cell housing/well in the implantable device are bonded Individual Cree UltraThin blue LED and Rohm semiconductor PicoLED series orange LEDs. In alternative embodiments, different types and/or wavelengths of light sources may be used. The blue LEDs provide the optical “ON” signal (e.g., 2 s pulse) that turns on the step-function opsin, e.g., SOUL, leading to the elevated calcium levels in the engineered cells, as illustrates in plasmid (1) of FIG. 2, which in turn lead to the production of the therapeutic agents by the engineered cell, as illustrated in plasmid (2) of FIG. 2. The orange LED will provide an optical “OFF” signal (e.g., 2 s pulse) that closes the step-function opsin. By tuning the interval between the ON and OFF signal (Δt), one can control the intracellular Ca++ levels and thus the therapeutic dose with a power savings of approximately 50× compared to traditional optogenetic control. To make the dose levels precise, optoelectronics are integrated in the carrier and used to track the fluorescent reporters associated with each therapy. In an illustrative embodiment, the same blue LED used for the ON signal can be used as the excitation light source to track GFP* fluorescence.

In another illustrative embodiment, fluorescence measurements can be made by integrating a green emission light collected by the photodiode over the blue light stimulation block. The LED and photodiode performance can be measured in vitro by comparing fluorometry data to ground truth microscopy data that will measure LED timing, intensity, and fluorescence. In one implementation, lifetime testing can include soaking the encapsulated LEDs in phosphate buffered saline at 37° C. for two months. For in vivo experiments to test photometry, fluorescent microspheres are encapsulated in the chassis and the fluorescence levels from the carrier implanted subcutaneously can be measured.

The GFP* emission is not expected to interfere with the optogenetic system activation state since the emission light is approximately 10⁶ times weaker than the LEDs. Additionally, the feedback controller will account for any non-idealities by adjusting Δt to maintain a desired production setpoint. In an alternative embodiment, an alternative destabilized fluorescent protein such as DsRed-Express that can be excited using the orange “OFF” LED is used, such that any issues regarding cross-talk between the fluorophore and the control signals can be solved.

### In Vivo Testing of Drug Delivery

It is important to validate engineered cells in vivo to verify that they display or execute to the proper engineered functions. In one implementation, an experimental group (e.g., mice) has encapsulated engineered cells implanted via an incision/suture procedure, and subsequently have the engineered cells turned on/off using the optogenetic system. Control groups include a first group implanted with encapsulated engineered cells with no optogenetic activation, a second group implanted with just the materials with no cells, and a third group implanted with triazole-thiomorpholine dioxide (TMTD) modified alginate.

When compared to the experimental group, the group implanted with engineered cells with no optogenetic activation allows one to verify the ability of the system to trigger production of the therapeutic protein. Also, when compared to the experimental group and first control group, the second group implanted with just the material allows one to determine whether the optogentic system is “leaking” and producing the therapeutics without being triggered. TMTD modified alginate is a material that is known to not evoke an immune response in mice, and can thus be used as a negative control when looking at the immune response that the material would evoke.

The above-discussed procedure is used to validate the ability of the system to controllably deliver therapeutics, e.g., GLP-1 and Orexin-A, in vivo. Specifically, engineered cells are encapsulated in an immune-protective material and implanted in the subcutaneous space of the test subjects and the skin sutured shut. In vivo light exposure and assaying for protein production is performed. The therapeutics (e.g., GLP-1 and Orexin A in one embodiment) are assayed for via ELISA, while GFP* is assayed via microscopic imaging (e.g., simple fluorescence microscopy, or in vivo imaging system (IVIS)). Implanted engineered cells are exposed to activating light through the skin of the test subject in varying patterns to demonstrate control over expression patterns. Various time points after light exposure are taken to determine the rate that the therapeutics are secreted once the cells are turned on. At each time point, blood samples were taken, along with IVIS fluorescence images. Blood samples are assayed for therapeutics, and the IVIS images are used to quantify GFP* production. ELISA and fluorescence data is compared to calibrate how much fluorescence correlates to a quantity of therapeutic produced. Immune response to implanted encapsulated cells is also measured. Specifically, immune response to the implanted material can be determined by simple microscopy after explant (fibrosis will appear as a layer of biological deposition on the implant if it evoked an immune response). Additionally, immune cell phenotyping can be performed at the implant site to identify any immune cells that are present.

### Engineered Cells Using HEK293T Cells

To further expand the horizon of the present invention, engineered cells with optogenetic system other than the engineered cells described above are designed and produced. HEK293T cells are genetically modified with plasmids for expression of therapeutic agents. HEK293 cells are immortalized human embryonic kidney cells, and HEK293T cell line is a derivative human cell line that expresses a mutant version of the SV40 large T antigen, generated by stable transfection of the HEK 293 cell line with a plasmid encoding a temperature-sensitive mutant of the SV40 large T antigen.

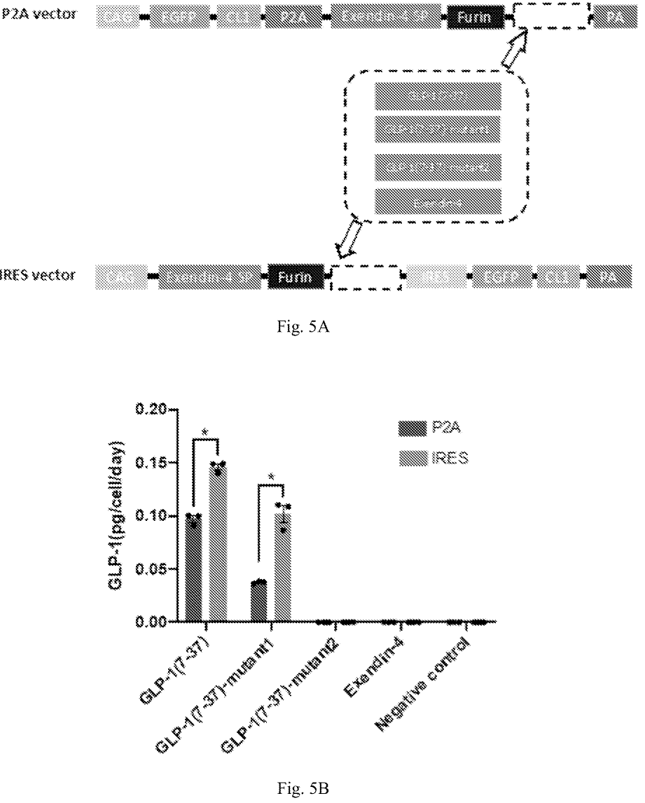

As shown in FIGS. 5A-5B, GLP-1 expressing plasmids under both P2A and IRES architectures are engineered to ensure 1:1 GLP-1 to EGFP expression. Multiple combinations of secretion signal peptides and GLP-1 gene were tested to maximize GLP-1 expression. In particular, as shown in FIG. 5A, P2A vector and IRES vector each contains a CAG of SEQ ID No: 1, a EGFP of SEQ ID No: 7, a CL1 of SEQ ID No: 5, a P2A of SEQ ID No: 3, an Exendin-4 SP, and a PA of SEQ ID No: 6, respectively. GLP-1 and its two mutant forms having SEQ ID Nos. 8-10 and Exedin-4 of SEQ ID No: 11 are included in the two vectors as well. FIG. 5A illustrates alignment of sequence domains. CAG: CMV enhancer, chicken beta-Actin promoter; CL1: degron; P2A: self-cleaving peptide; IRES: internal ribosome entry site; Exendin-4 SP: exendin-4 signal peptide; Furin: furin cleavage site; GLP-1(7-37)-mutant1: GLP-1(7-37)Gly8; GLP-1(7-37)-mutant2: GLP-1(7-37)Gly8/Gln26/Asp34; PA, poly(A) signal.

HEK 293T cells were separately transfected with two GLP-1 expressing vectors illustrated in FIG. 5A and the media changed 24 hrs post-transfection. GLP-1 value in media were assayed with ELISA 24 hrs later, (N=3).

Through transient transfection of these plasmids in HEK293T cells, it is shown that engineered cells transfected with IRES vector demonstrates a stronger GLP-1 production. Thus, an optimal secretion signal (exendin-4 signal peptide) and the IRES system are preferred architectures over the P2A system in driving peptide production, as illustrated in FIG. 5B.

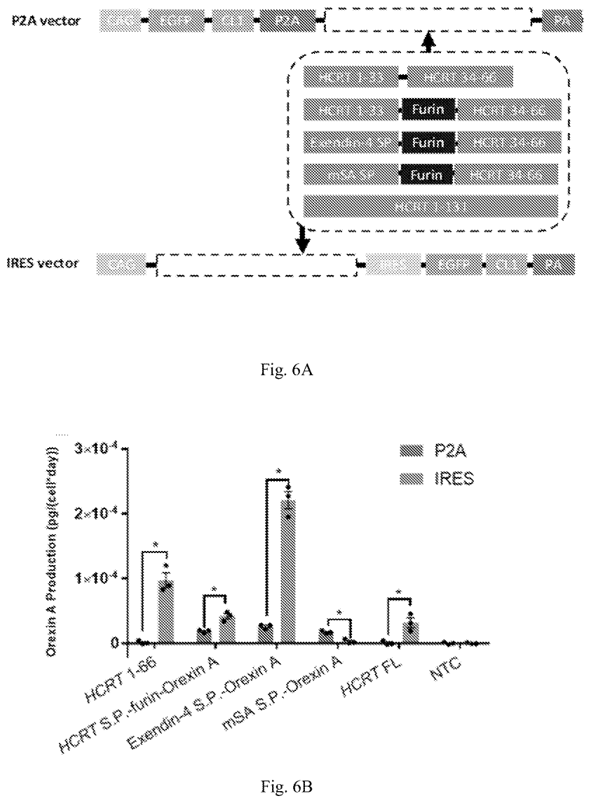

In another illustrative embodiment, plasmids expressing orexin-A under P2A and IRES architectures are used to transfected HEK293T cells to ensure 1:1 orexin-A and EGFP expression. As shown in FIG. 6A, multiple combinations of secretion signal peptides and orexin A gene were tested to maximize orexin A expression. In particular, five different sequences SEQ ID Nos: 12-16, encoding orexin A or its fragments, are used as alternatives in P2A and IRES vectors respectively. Construction of two vectors are shown in FIG. 6A: Orexin-A constitutes amino acids 34-66 of hypocretin (HCRT) while HCRT1-33 is the original secretion signal of orexin-A. CAG: CMV enhancer, chicken beta-Actin promoter; CL1: degron; P2A: self-cleaving peptide; IRES: internal ribosome entry site. Exendin 4 SP: secretion signal peptide of exendin-4. mSA SP: modified human albumin signal peptide. Furin: furin cleavage site. HCRT 1-131: full length gene of hypocretin.

FIG. 6B shows orexin-A production by HEK 293T cells 24 hours after transfection of expression plasmids. In particular, through transient transfection of these plasmids in HEK293T cells, the present invention identified an optimal secretion signal (exendin-4 signal peptide) and the IRES system as the lead architecture over the P2A system in driving peptide production, similar to the GLP-1 system. It is also validated that the cellular capacity of synthetic orexin A production was significantly lower than the projected goal (2.4e⁻⁴ pg/cell/day) and that there is no significant intracellular accumulation of orexin-A.

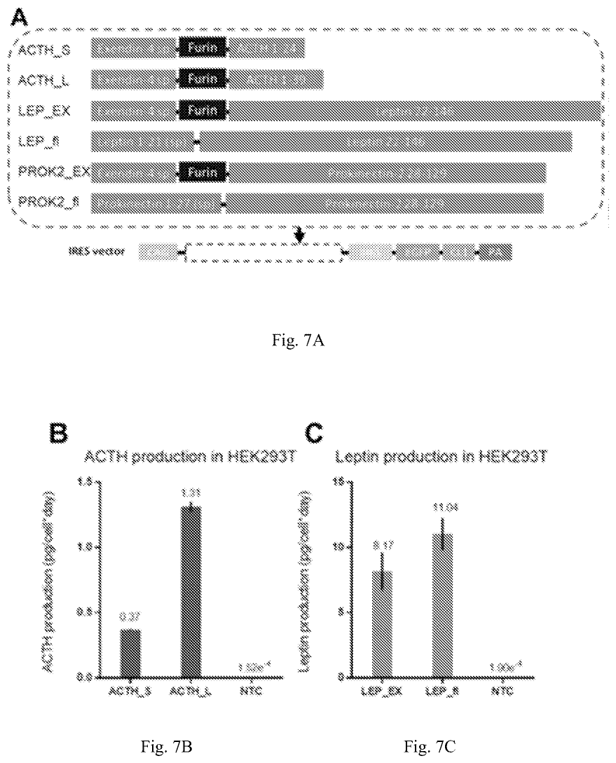

FIGS. 7A-C show two additional neuropeptides: leptin and adrenocorticotropic hormone (ACTH), which were thus tested in the lead configurations identified above, namely IRES architecture with exendin-4 signal peptide as secretion signal. Two versions of secretion signal-peptide gene combination were cloned into the IRES system plasmid for each new peptide, as shown in FIG. 7A. In particular, for ACTH, SEQ ID Nos: 17-18 were each individually cloned into the IRES vector, and for Leptin, SEQ ID Nos: 19-20 were each individually closed into the IRES vector.

Through transient transfection in HEK293T cells, we were able to achieve an ACTH secretion rate of 1.3 pg/cell/day as shown in FIG. 7B and a leptin secretion rate of 11 pg/cell/day as shown in FIG. 7C.

### Engineered Cells Using ARPE-19 Cells

In another illustrative embodiment, ARPE-19 cells are genetically modified with plasmids expressing a therapeutic agent and a report agent at 1:1 ratio.

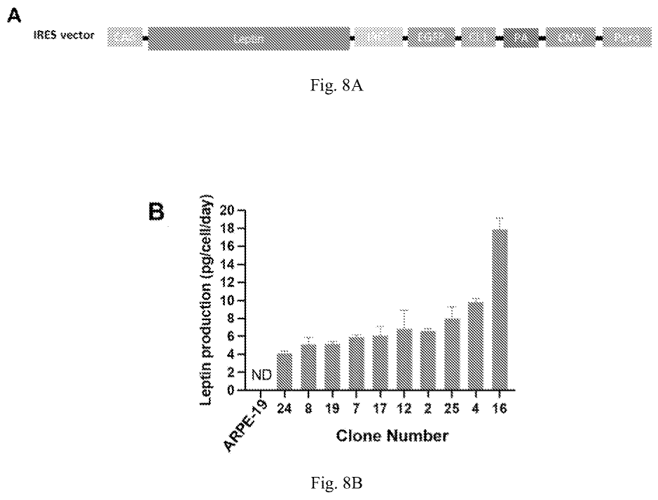

As shown in FIG. 8A, ARPE-19 cells are genetically engineered to stably express a leptin and EGFP in an IRES based co-expression system. Clonal cell lines were then made from those transfected cells by plating a single cell per well and allowing the cells to grow out in a generally used cell medium. Leptin secretion from the cells was then assayed by ELISA and a clonal line which produces 18 pg/cell/day of leptin was identified. It should be noted that the IRES vector used for ARPE-19 cells includes a CMV sequence of SEQ ID No: 23 and a Puro sequence of SEQ ID No: 24 downstream of PA.

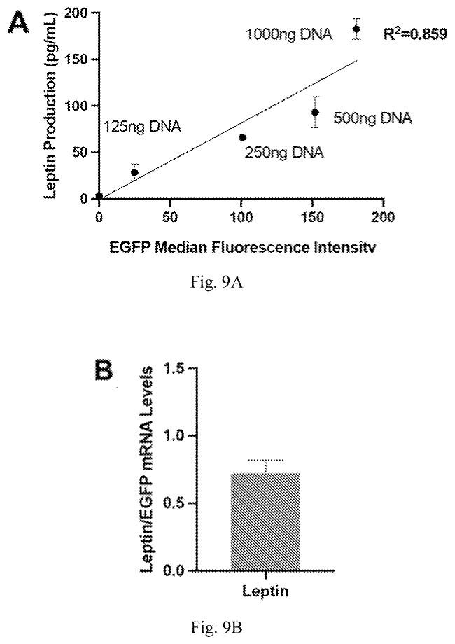

To demonstrate 1:1 production of leptin and EGFP, ARPE-19 cells were transfected with increasing amounts of the same DNA used to make the stable cell line. Leptin production from the cells was measured by ELISA and EGFP expression was measured by flow cytometry. Leptin and EGFP expression correlated linearly with an R value of 0.859, as shown in FIG. 9A. Additionally, leptin and EGFP mRNA levels in ARPE-19 cells transfected with the same DNA were assayed by qPCR. The two mRNA levels showed a near 1:1 ratio (0.72:1), which was within the variation expected for the assay as shown in FIG. 9B.

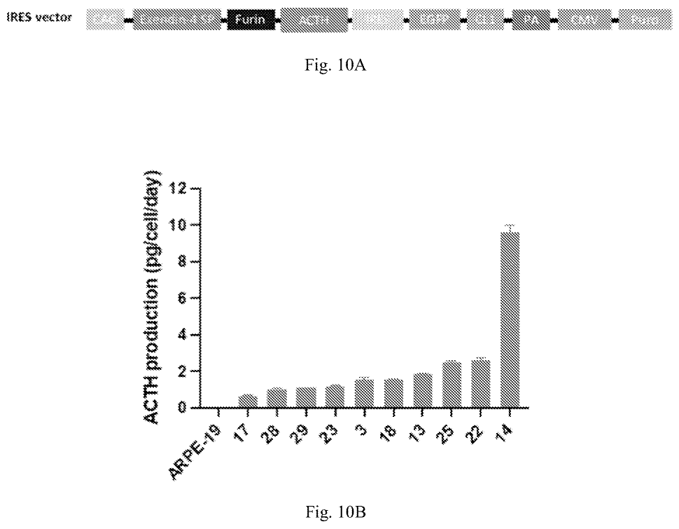

In another embodiment, ARPE-19 cells are genetically engineered with a plasmid having IRES vector architecture as shown in FIG. 10A to stably express ACTH and EGFP. ACTH having SEQ ID No: 18 is used. Similarly clonal cell lines were created and assayed for ACTH secretion. In particular, the cells were then plated to single cell/well density and allowed to grow out. One expanded ACTH production from the clonal cell lines was assayed by ELISA. Data is shown in pg/cell/day in FIG. 10B. A clonal cell line which produces 10 pg/cell/day of ACTH was identified, according to FIG. 10B.

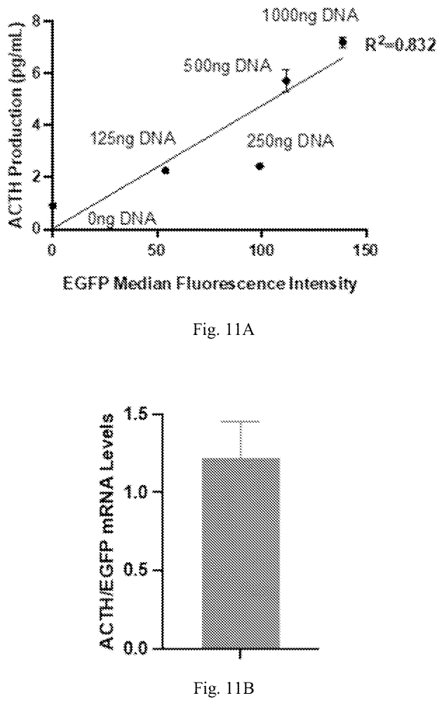

The same methods as described above were used to demonstrate 1:1 production of ACTH and EGFP. In particular, ARPE-19 cells were transfected with increasing amounts of the plasmid and protein expression was measured. EGFP was assayed by flow cytometry and ACTH was assayed by ELISA. ACTH and EGFP protein production linearly correlated with an R value of 0.832, as shown in FIG. 11A. mRNA levels of ACTH and EGFP quantified using qPCR showed a near 1:1 ratio (1.21:1) that was within the variation expected for the assay, as shown in FIG. 11B.

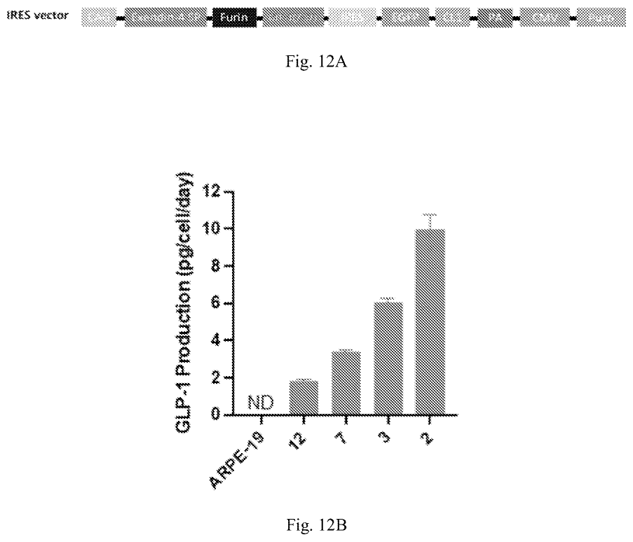

In another embodiment, same process was used to create ARPE-19 clonal cell lines expressing GLP-1 and EGFP, with the IRES architecture as shown in FIG. 12A. In particular, GLP-1 of SEQ ID No: 8 is linked downstream to Exendin-4 SP and Furin. FIG. 12A shows a schematic of GLP-1 expression plasmid: CAG: CMV enhancer, chicken beta-Actin promoter; CL1: degron; IRES: internal ribosome entry site; Exendin-4 SP: exendin-4 signal peptide; Furin: furin cleavage site; GLP-1(7-37); Glucagon Like Peptide 1. Later, same to above, ARPE-19 cells were stably transfected with the plasmid shown in FIG. 12A. The cells were then plated to single cell/well density and allowed to grow out. Once expanded GLP-1 production from the clonal cell lines was assayed by ELISA. Data is shown in pg/cell/day. A clonal cell line was identified that produces 10 pg/cell/day of GLP-1 in FIG. 12B.

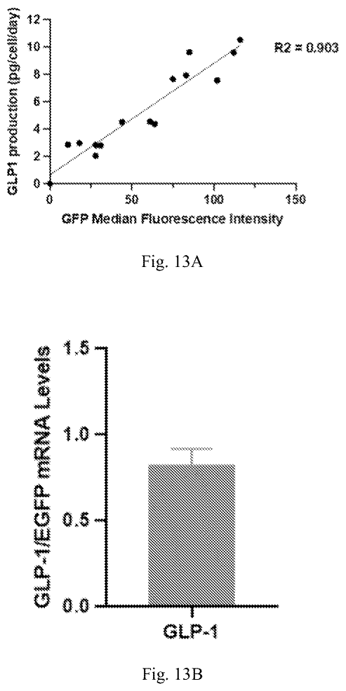

FIGS. 13A-B show correlation of GFP and GLP-1 expression on the protein level. AREP-9 cells were transfected with increasing amounts of the plasmid depicted in FIG. 12A, and protein expression was measured. EGFP was assayed by flow cytometry and GLP-1 was assayed by ELISA. GLP-1 and EGFP protein production correlated linearly with an R value of 0.903, as shown in FIG. 13A. Then, APRE-19 cells were transfected with the plasmid depicted in FIG. 12A and mRNA levels of GLP-1 and EGFP were quantified using qPCR. Data is plotted as the ratio of GLP-1 mRNA to EGFP mRNA. Levels of the two mRNA transcripts were near 1:1 (0.80:1), according to FIG. 13B.

### Optogenetic System Engineering

As illustrated in FIG. 2, plasmid (2) encodes therapeutic agents and reporter agent, while plasmid (1) encodes light sensing proteins of an optogenetic system driven by a CAG promoter, to enable constitutive expression of the optogentic system, e.g., production of opsin SOUL.

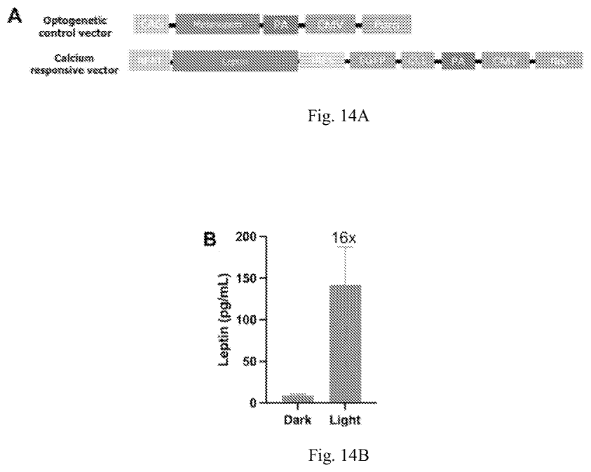

In one embodiment, melanopsin is used as the light sensing protein for optogenetic system. As shown in FIG. 14A, plasmids (1) containing optogenetic control vector includes a CAG of SEQ ID No: 1, a melanopsin of SEQ ID No: 21, PA of SEQ ID No: 6, CMV of SEQ ID No: 23, and Puro of SEQ ID No: 24. Plasmids (2) containing calcium responsive vector, which expresses the therapeutic agents and reporter agent, includes NFAT promoter of SEQ ID No: 2, Leptin of SEQ ID No: 20, IRES of SEQ ID No: 4, EGFP of SEQ ID No: 7, CL1 as degron, PA of SEQ ID No: 6, CMV of SEQ ID No: 23, and Neo of SEQ ID No: 2. The NFAT promoter is a light sensing protein activated promoter, indirectly driven by the melanopsin or SOUL via the calcium level in the engineered cells, as disclosed in FIG. 2. Plasmids (1) and (2) were both transfected into ARPE-19 cells. Thus, in the transfected ARPE-19 cells, plasmids (1) expressing the melanopsin based optogenetic system driving production of leptin by plasmids (2) under a similar architecture.

Transfected ARPE-19 cells were left in the dark or exposed to blue light 5 s ON 10 s OFF for 24 hours before the media was collected and assayed for leptin by ELISA. As shown in FIG. 14B, leptin production increased 16-fold in response to light.

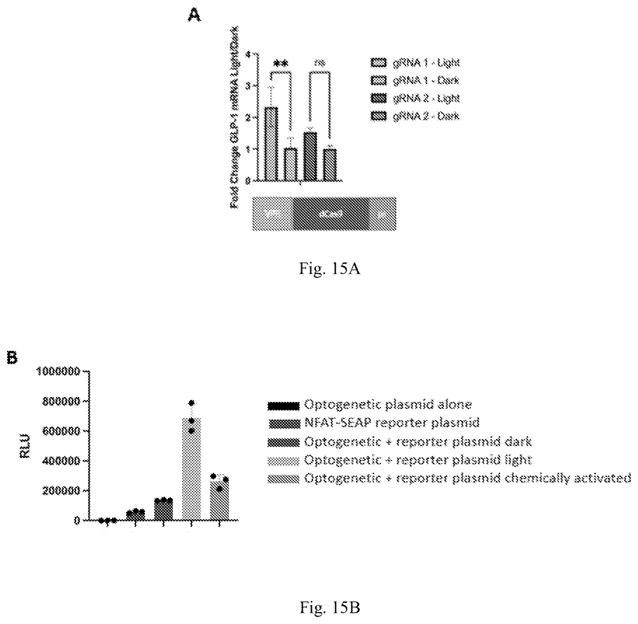

In one embodiment, an optogenetic system based on a photoswitchable dCas9 was constructed and tested. To test the system, HEK293 Ts were transiently transfected with the photo switchable dCas9 having SEQ ID No: 22, NFAT-GLP1 expressing GLP-1, and a sgRNA targeting the NFAT promoter. After 24 hours, cells were exposed to blue light for 24 hours. Cells were then harvested and processed for RT-qPCR. As shown in FIG. 15A, cells exposed to blue light for 24 hours showed a 2-fold increase in GLP-1 mRNA over cells left in the dark.

In an alternative embodiment, an optogenetic system using human melanopsin (hMelanopsin) having SEQ ID No: 21 were constructed to increase intracellular calcium levels and drive expression of a transgene SEAP having SEQ ID NO: 28 under the NFAT promoter.

Exposing HEK293 Ts transiently transfected with plasmid containing hMelanopsin and plasmid containing NFAT-SEAP to blue light over the course of 24 hours increased expression of a reporter protein SEAP 5-fold over SEAP expression by cells cultured in the dark, as shown in FIG. 15B

In another embodiment, SOUL or mutant SOUL are used as the light sensing protein for the optogenetic system. The present invention constructs plasmids expressing the channel rhodopsin mutant SOUL and a reporter plasmid with expressing SEAP driven by NFAT to create an optogenetically activated transcription system in HEK293T cells.

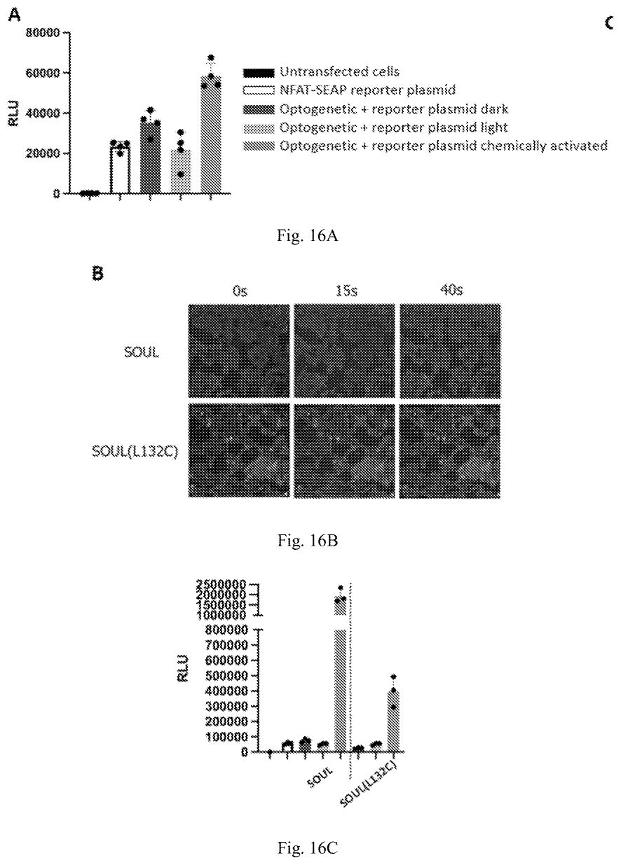

However, as shown in FIG. 16A, in initial testing of the system, it is found that this system did not work to drive protein production. In particular, over the course of 24 hours, SEAP expression from HEK293 Ts transiently expressing SOUL of SEQ ID No: 26 and SEAP of SEQ ID No: 28 driven by NFAT promoter of SEQ ID No: 2 from cells reflects that SEAP expression in blue light condition was even lower than that of in dark condition.

Based on the hypothesis that SOUL was letting in insufficient calcium ions to drive transcription from NFAT promoter, a novel light responsive ion channel SOUL(L132C) that includes a mutation found to increase calcium influx by similar ion channels was constructed. Exposing HEK293T cells transiently expressing SOUL of SEQ ID No: 26 or SOUL(L132C) of SEQ ID No: 27 to blue light led to an increase in intracellular calcium in cells expressing SOUL(L132C), but not in cells expressing SOUL, as shown in FIG. 16B.

Therefore, HEK293T cells co-expressing SOUL, or SOUL(L132C) and NFAT promoter driving the reporter protein were tested for SEAP expression. As shown in FIG. 16C, light exposure induced a 2-fold increase in protein expression from the SOUL(L132C) system. This is consistent with the intracellular data that the SOUL based system did not induce an increase in protein expression.

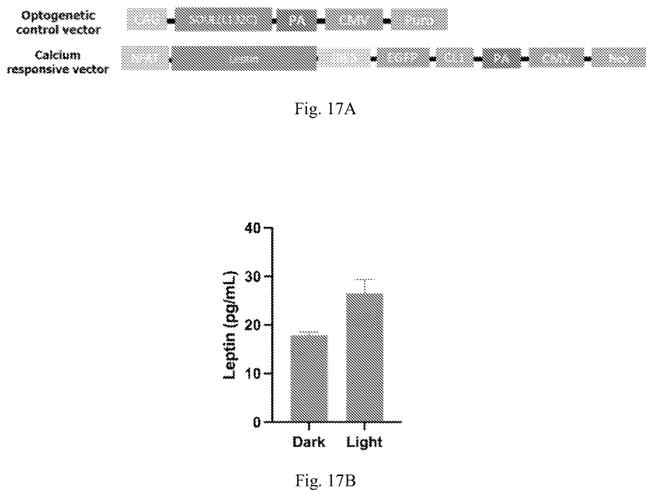

In alternative embodiment, ARPE-19 cells expressing the low-light sensitive SOUL(L132C) optogenetic system driving expression of leptin were also created. In particular, ARPE-19 cells were transfected with plasmids containing an optogenetic control vector having SOUL (L132C) of SEQ ID No: 27, and plasmids containing calcium response vector having leptin of SEQ ID No. 20. The construction of two vectors are reflected in FIG. 17A.

ARPE-19 cells were stably transfected with the plasmids shown in FIG. 17A. The cells were left in the dark or exposed to blue light 10 s ON 10 m OFF for 24 hours before the media was collected and assayed for leptin by ELISA. As shown in FIG. 17B, leptin production increased 1.5-fold in response to the light.

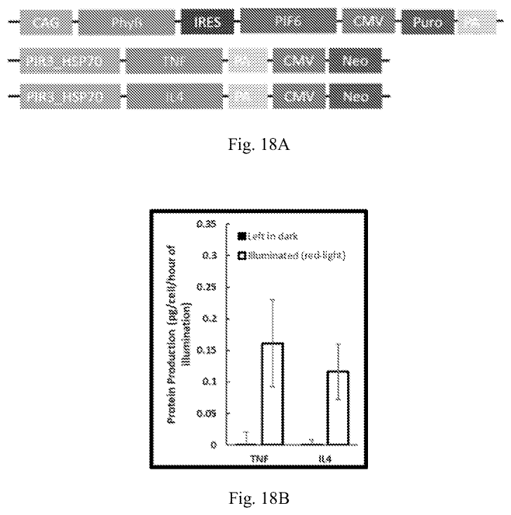

In an alternative embodiment of the optogenetic system, instead of SOUL or melanopsin, PhyB-PIF6 complex is used as light sensing proteins for the optogenetic system. In particular, as shown in FIG. 18A, ARPE-19 cells are transfected with plasmids of IRES architecture containing PhyB DNA having SEQ ID No: 33 and PIF6 DNA having SEQ ID No: 35. The ARPE-19 cells transfected with these plasmids express PhyB protein and PIF6 protein (Phyb/PIF6 complex) as the optogenetic system, which can be trigger by red light. The ARPE-19 cells are also transfected with plasmids expressing TNF having SEQ ID No: 31 and IL4 having SEQ ID No: 32, which are either separately or commonly driven by a PIR3_HSP70 min promoter having SEQ ID No: 30. The PIR3_HSP70 min promoter is a light sensing protein activated promoter, activated by the Phyb/PIF6 complex. ARPE-19 cells engineered with these plasmids create stable cell lines expressing TNF and IL4 in response to red light. Cells were then activated with red light or left in the dark and the TNF and IL4 productions were measured by ELISA.

FIG. 18A shows a schematic of plasmids. CAG: CMV enhancer, chicken beta-Actin promoter; PhyB: Phytochrome B; IRES: internal ribosome entry site; PIF6: Phytochrome Interacting Factor 6; PA, poly(A) signal TNF: tumor necrosis factor; IL4: Interleukin 4 Puro: puromycin resistance; Neo: neomycin resistance.

FIG. 18B shows stably engineered ARPE-19 cells were plated and exposed to light or kept in the dark. Media was assayed for TNF and IL4 by ELISA to calculate production. As it can be seen in FIG. 18B, comparing to the engineered cells left in dark, the Phyb/PIF6 is triggered by the red light in the ARPE-19 cells, which in turn activates the PIR3_HSP70 min promoter driving the expression of TNF and IL4.

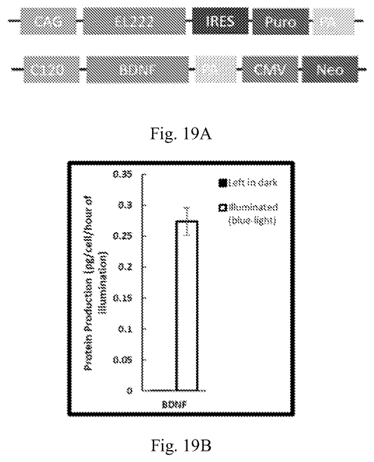

In an alternative embodiment of the optogenetic system, EL222 is used as a light sensing protein for the optogenetic system and the C120 promoter is used as a light sensing protein activated promoter, activated by the EL222 and driving the expression of BDNF. In particular, as shown in FIG. 19A, plasmids having IRES architecture containing EL222 DNA of SEQ ID No: 35 driven by a CAG promoter is used for transfecting ARPE-19 cells. The ARPE-19 cells are also transfected with plasmids containing a C120 promoter of SEQ ID No: 36, driving the expression of BDNF encoded by a DNA sequence of SEQ ID No: 37. ELISAs were performed measure the production rate of BDNF from ARPE-19 cells engineered with expression plasmids. Data is shown in pg/cell/hour of illumination.

FIG. 19A shows a schematic of expression plasmids. CAG: CMV enhancer, chicken beta-Actin promoter; EL222: light-oxygen-voltage domain and LuxR-type helix-turn-helix DNA-binding domain; PA, poly(A) signal; BDNF: Brain derived neurotrophic factor; Puro: puromycin resistance; Neo: neomycin resistance. FIG. 19B shows stably engineered ARPE-19 cells were plated and exposed to blue light or kept in the dark. Media was assayed for BDNF by ELISA to calculate production. As shown in FIG. 19B, the expression of BNNF by engineered ARPE-19 cells was around 0.275 pg/cell/hour of illumination when exposed to the blue light, as compared to nearly 0 pg/cell/hour of illumination when left in dark.

Although plasmids are used in aforementioned disclosure for transfection of the cells or introduction of transgenes into the cells, it should be noted that other vectors commonly used for cell transfection, e.g., virus, can be used as alternative embodiments.

It should also be noted that, although transgenes of the light sensing proteins, transgenes of the therapeutic agents/reporter agents, transgenes of the dCas9 protein, and transgenes of the iCaspase9 protein, may be transfected into the cells using separate vectors, those transgenes may be integrated into one or more vectors which are then used for cell transfection.

### Circadian Rhythm Control

In one exemplary implementation, the system can be used to help control the circadian rhythm of the subject in which the system is implanted. For example, the system can be used to accelerate human adaptation to a new time zone or work schedule by synergistically shifting central and peripheral circadian clocks. While various examples and implementation details are provided herein with respect to control of circadian rhythm, it is to be understood that the system is not limited to circadian rhythm applications. Rather, as discussed herein, the proposed implantable cell generation system can be used to provide pain relief, fight diseases, cure disorders, provide immune response control, treat infertility, etc.

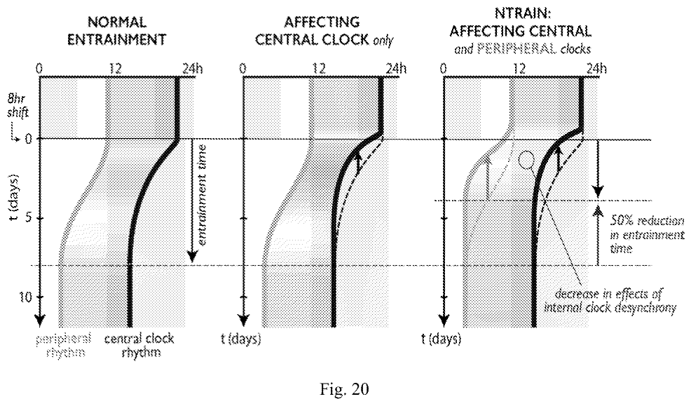

In one exemplary embodiment of multiple therapeutic delivery, multi-clock targeting with precision timing for circadian rhythm regulation can be performed by the system. For example, in the circadian rhythm example, by targeting both the central and peripheral clocks, the hybrid bioelectronics system of the present invention provides synergistic effects towards enhanced entrainment. However, because the same therapy applied during different phases of a circadian rhythm can have both phase-advancing or phase-delaying effects, it is important to validate therapeutic efficacy in terms of its administration window. The system uses phase response curves (i.e., the phase shift induced by therapy as a function of the phase of delivery), combined with real time sensing of internal body temperature and/or commercial off the shelf (COTS) wearable sensors to inform actuation-timing for most effective delivery of therapies. FIG. 20 depicts phases of peripheral and central clocks in response to an 8 hr shift, for normal entrainment (left), providing therapy affecting only the central clock (middle), and the approach of the hybrid bioelectronics system of the present invention (right) with therapy targeting both central and peripheral clocks in accordance with an illustrative embodiment. In FIG. 20, the fill color green represents normal phase relationship, and red represents misaligned phases. The system of the present invention rises above the current state-of-the-art in circadian rhythm management because it delivers a personalized therapy with precision dosing and timing for maximum efficacy. This is not possible with single-dose approaches that act only on sleep/wake rhythms.

In the illustrative embodiment for the circadian rhythm application, the therapeutics targeted for production and delivery by the engineered cells are GLP-1 and Orexin A. Unlike recombinant variants, GLP-1 and Orexin A have short metabolic half-lives (GLP-1, 4.6-7.1 min; Orexin A, 27 min), making their use for entrainment more effective. Such half-lives are long enough to reach target tissues, short enough to have a precisely timed phase-shifting action, and are known to readily cross the blood-brain barrier, exhibiting potent actions on the brain when peripherally administered.

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

1. An engineered cell expressing a therapeutic agent and a reporter agent, the engineered cell comprising:
a first transgene comprising a light sensing protein DNA encoding a light sensing protein;
a second transgene comprising a light sensing protein activated promoter, a therapeutic agent DNA encoding a therapeutic agent, and a reporter DNA encoding a reporter;
a third transgene comprising the light sensing protein activated promoter and a dCas9 DNA encoding a dCas9 protein; and
a fourth transgene comprising a CASP9 DNA encoding an iCaspase9 protein leading to cell death;
wherein the light sensing protein, when receiving a light, actives the light sensing protein activated promoter;
wherein the light sensing protein activated promoter, when activated, is configured to start production of the therapeutic agent and the reporter agent; and
wherein the therapeutic agent and the reporter agent are configured to be expressed at a substantially fixed ratio.

2. The engineered cell according to claim 1, wherein the light sensing protein comprises at least one of a step-function opsin (SOUL), a mutant of SOUL, a melanopsin, a PhyB/PIF6 complex, and a EL222.

3. The engineered cell according to claim 2, wherein the light sensing protein DNA sequence comprises at least one of SEQ ID Nos: 21, 26-27, and 33-35 or an equivalent DNA of at least one of SEQ ID Nos: 21, 26-27, and 33-35.

4. The engineered cell according to claim 1, wherein the light sensing protein activated promoter comprises one of SEQ ID Nos: 2, 30, and 36 or an equivalent DNA of one of SEQ ID Nos: 2, 30, and 36.

5. The engineered cell according to claim 1, wherein the therapeutic agent DNA comprises one of SEQ ID Nos: 8-20, 28, 31, 32, and 37 or an equivalent DNA of one of SEQ ID Nos: 8-20, 28, 31, 32, and 37.

6. The engineered cell according to claim 1, wherein the reporter agent DNA comprises SEQ ID No: 7 or an equivalent DNA of SEQ ID No: 7.

7. The engineered cell according to claim 1, wherein the second transgene comprises an IRES DNA or a P2A DNA locates between the therapeutic agent DNA and the reporter agent DNA.

8. The engineered cell according to claim 7, wherein the IRES DNA comprises SEQ ID No: 4 or an equivalent DNA of SEQ ID No: 4, and the P2A DNA comprises SEQ ID No: 3 or an equivalent DNA of SEQ ID No: 3.

9. The engineered cell according to claim 1, wherein the engineered cell is an ARPE-19 cell transfected by at least one of the first, second, third, and fourth transgenes.

10. The engineered cell according to claim 1, wherein the engineered cell is a HEK293T cell transfected by at least one of the first, second, third, and fourth transgenes.

11. An engineered cell expressing a therapeutic agent and a reporter agent, the engineered cell comprising:
a first transgene comprising a light sensing protein DNA sequence encoding a light sensing protein; and
a second transgene comprising a light sensing protein activated promoter, a therapeutic agent DNA encoding a therapeutic agent, and a reporter agent DNA encoding a reporter agent.

12. The engineered cell according to claim 11, wherein the light sensing protein activated promoter is configured to control the expression of the therapeutic agent and the reporter agent.

13. The engineered cell according to claim 12, wherein the light sensing protein comprises at least one of a step-function opsin (SOUL), a mutant of SOUL, a melanopsin, a PhyB/PIF6 complex, and a EL222.

14. The engineered cell according to claim 12, wherein the light sensing protein activates the light sensing protein activated promoter which drives production of the therapeutic agent and the reporter agent in the engineered cell.

15. The engineered cell according to claim 11, wherein the light sensing protein DNA sequence comprises at least one of SEQ ID Nos: 21, 26-27, and 33-35 or an equivalent DNA of at least one of SEQ ID Nos: 21, 26-27, and 33-35.

16. The engineered cell according to claim 11, wherein the light sensing protein activated promoter comprises one of SEQ ID Nos: 2, 30 and 36 or an equivalent DNA of one of SEQ ID Nos: 2, 30, and 36.

17. The engineered cell according to claim 11, wherein the therapeutic agent and the reporter agent are expressed at a substantially fixed ratio.

18. The engineered cell according to claim 11, wherein the therapeutic agent DNA comprises one of SEQ ID Nos: 8-20, 28, 31, 32, and 37 or an equivalent DNA of SEQ ID Nos: 8-20, 28, 31, 32, and 37.

19. The engineered cell according to claim 11, wherein the reporter agent DNA comprises SEQ ID No: 7 or an equivalent DNA of SEQ ID No: 7.

20. The engineered cell according to claim 11, wherein the second transgene comprises an IRES DNA or a P2A DNA locates between the therapeutic agent DNA and the reporter agent DNA.

21. The engineered cell according to claim 20, wherein the IRES DNA comprises SEQ ID No: 4 or an equivalent DNA of SEQ ID No: 4, and the P2A DNA comprises SEQ ID No: 3 or an equivalent DNA of SEQ ID No: 3.

22. The engineered cell according to claim 11, further comprising a third transgene comprising a light sensing protein activated promoter and a dCas9 DNA encoding a dCas9 protein.

23. The engineered cell according to claim 11, further comprising a fourth transgene comprising a CASP9 DNA encoding an iCaspase9 protein leading to cell death.

24. The engineered cell according to claim 11, wherein the engineered cell is an ARPE-19 cell transfected by the first and second transgenes.

25. The engineered cell according to claim 11, wherein the engineered cell is a HEK293T cell transfected by the first and second transgenes.

26. A method for producing an engineered cell expressing a therapeutic agent and a reporter agent, the method comprising:
transfecting a cell with a first transgene comprising a light sensing protein DNA sequence encoding a light sensing protein; and
transfecting the cell with a second transgene containing a light sensing protein activated promoter, a therapeutic agent DNA encoding a therapeutic agent, and a reporter agent DNA encoding a reporter agent.

27. The method for producing an engineered cell according to claim 26, further comprising:
transfecting the cell with a third transgene comprising the light sensing protein activated promoter and a dCas9 DNA encoding a dCas9 protein.

28. The method for producing an engineered cell according to claim 27, further comprising:
transfecting the cell with a fourth transgene containing a CASP9 DNA encoding an iCaspase9 protein leading to cell death.

29. The method for producing an engineered cell according to claim 26, wherein the light sensing protein comprises at least one of a step-function opsin (SOUL), a mutant of SOUL, a melanopsin, a PhyB/PIF6 complex, and a EL222.

30. The method for producing an engineered cell according to claim 26, wherein the light sensing protein activated promoter is one of a NFAT promoter comprising SEQ ID No: 2 or an equivalent DNA of SEQ ID No: 2, a PIR3_HSP70 min promoter comprising SEQ ID No: 30 or an equivalent DNA of SEQ ID No: 30, and a C120 promoter comprising SEQ ID No: 36 or an equivalent DNA of SEQ ID No: 36.

31. The method for producing an engineered cell according to claim 26, wherein the therapeutic agent and the reporter agent are expressed at a substantially fixed ratio.

32. The method for producing an engineered cell according to claim 26, wherein the therapeutic agent DNA comprises one of SEQ ID Nos: 8-20, 28, 31, 32, and 37 or an equivalent DNA of one of SEQ ID Nos: 8-20, 28, 31, 32, and 37.

33. The method for producing an engineered cell according to claim 26, wherein the reporter agent DNA comprises SEQ ID No: 7 or an equivalent DNA of SEQ ID No: 7.

34. A transgene configured to produce a therapeutic agent and a reporter agent at a substantially fixed ratio, the transgene comprising following formula:
(Light sensing protein activated promoter)-(B)ₙ-(Therapeutic agent DNA)-(B)ₘ-(IRES DNA)-(B)ₒ-(Reporter agent DNA), or
(Light sensing protein activated promoter)-(B)ₙ-(Reporter agent DNA)-(B)ₘ-(P2A DNA)-(B)ₒ-(Therapeutic agent DNA),
wherein:
B is independent deoxyribonucleotide; and
n, m, o are integer numbers, wherein n, m, and o are identical to or different from each other.

35. The plasmid according to claim 34, wherein the light sensing protein activated promoter is one of a NFAT promoter comprising SEQ ID No: 2 or an equivalent DNA of SEQ ID No: 2, a PIR3_HSP70 min promoter comprising SEQ ID No: 30 or an equivalent DNA of SEQ ID No: 30, and a C120 promoter comprising SEQ ID No: 36 or an equivalent DNA of SEQ ID No: 36.

36. The plasmid according to claim 35, wherein the IRES DNA comprises SEQ ID No: 4 or an equivalent DNA of SEQ ID No: 4, wherein P2A DNA comprises SEQ ID No: 3 or an equivalent DNA of SEQ ID No: 3.

37. The plasmid according to claim 36, wherein therapeutic agent DNA comprises one of SEQ ID Nos: 8-20, 28, 31, 32, and 37 or an equivalent DNA of one of SEQ ID Nos: 8-20, 28, 31, 32 and 37.

38. The plasmid according to claim 37, wherein the reporter agent DNA comprises SEQ ID No: 7 or an equivalent DNA of SEQ ID No: 7.

39-40. (canceled)
